(19) United States
(12) Patent Application Publication
(10) Pub. No.: US 2024/0277749 A1
(43) Pub. Date: Aug. 22, 2024

(54) NEW FORMULATIONS COMPRISING AZACITIDINE

(71) Applicant: NANEXA AB, Uppsala (SE)

(72) Inventors: Anders JOHANSSON, Uppsala (SE); Mårten ROOTH, Uppsala (SE); Erik LINDAHL, Uppsala (SE); Joel HELLRUP, Uppsala (SE); David WESTBERG, Uppsala (SE)

(21) Appl. No.: 18/568,219

(22) PCT Filed: Jun. 10, 2022
(86) PCT No.: PCT/GB2022/051463

§ 371 Date: Dec. 7, 2023

(30) Foreign Application Priority Data
Jun. 10, 2021 (GB) 2108297.9
Dec. 8, 2021 (GB) 2117701.9

(51) Int. Cl. A61K 31/706; A61K 9/00; A61K 9/10; A61K 9/50; A61P 35/00; C23C 16/40; C23C 16/455

(52) U.S. Cl. CPC A61K 31/706; A61K 9/0019; A61K 9/10; A61K 9/501; A61K 9/5073; A61P 35/00; C23C 16/407; C23C 16/45555

(57) ABSTRACT

There is provided a pharmaceutical formulation that is useful in the treatment of myelodysplastic syndrome, comprising a plurality of particles suspended in an aqueous carrier system, which particles: (a) have a weight-, number-, or volume-based mean diameter that is between amount 10 nm and about 700 μm; and (b) comprise solid cores comprising azacitidine, or a pharmaceutically-acceptable salt thereof, coated, at least in part, by a coating of inorganic material comprising mixture of: (i) zinc oxide; and (ii) one or more other metal and/or metalloid oxides, wherein the atomic ratio ((i):(ii)) is between at least 1:6 and up to and including about 6:1. Said mixed oxide coated particles are preferably synthesized via a gas phase coating technique, such as atomic layer deposition. The formulation may provide for the delayed or sustained release of azacitidine to treat myelodysplastic syndrome without a burst effect.

### FIELD OF THE INVENTION

This invention relates to a new formulation for use in, for example, the field of drug delivery and in particular in the treatment of cancers, and in particular myelodysplastic syndrome (MDS).

### PRIOR ART AND BACKGROUND

The listing or discussion of an apparently prior-published document in this specification should not necessarily be taken as an acknowledgement that the document is part of the state of the art or common general knowledge.

MDS is a type of cancer in which immature blood cells in the bone marrow do not mature to become healthy blood cells. Problems with blood cell formation result in a combination of low red blood cell, platelet and white blood cell counts. Some types of MDS are also manifest by increased immature blood cell counts (blasts) in the bone marrow or blood.

The incidence of MDS is about 7 in 100,000 patients, with a typical age of onset of 70 years of age. There are sub-types of MDS, which are characterised according to specific changes in the blood cells and bone marrow.

Current treatments of MDS include supportive care (blood transfusions, medications known to increase red blood cell count and/or antibiotics), chemotherapy by way of cytotoxic agents, hematopoietic stem cell transplantation and/or combinations thereof.

The chemotherapeutic agent azacitidine (4-amino-1-®-D-ribofuranosyl-s-triazin-2(1H)-one; also known as 5-azacytidine or just ‘azacytidine’) is sold under the brand name Vidaza® and is commonly prescribed in the treatment of MDS. It is a chemical analogue of cytidine, a nucleoside in DNA and RNA.

At low doses, azacitidine's antineoplastic activity is believed to proceed by inhibition of DNA methyltransferase, causing hypomethylation of DNA. At high doses, it is believed to work by direct cytotoxicity to abnormal hematopoietic cells in the bone marrow through its incorporation into DNA and RNA, resulting in cell death.

Azacitidine's incorporation into RNA leads to the disassembly of polyribosomes, defective methylation and acceptor function of transfer RNA, and inhibition of the production of proteins. Its incorporation into DNA leads to covalent binding with DNA methyitransferases, which prevents DNA synthesis and subsequently leads to cytotoxicity.

Azacitidine is administered by injection subcutaneously or intravenously. These injections need to be administered frequently as they do not form a depot from which active ingredient leaches out over an extended period of time. In particular, the standard treatment cycle for azacitidine in the treatment of MDS is seven consecutive days of azacitidine injections at 28 day cycles.

In a condition like MDS, it would be advantageous to provide an extended release composition, in which the active ingredient is released at a desired and predictable rate in vivo following injection, in order to ensure a more optimal pharmacokinetic profile.

In the case of any sustained release composition, it is of critical importance that its release profile shows minimal initial rapid release of active ingredient, that is a large concentration of drug in plasma shortly after administration. Such a ‘burst’ release will result in unwanted, high concentrations active ingredient, and may be hazardous in the case of drugs that have a narrow therapeutic window or drugs that are toxic at high plasma concentrations. In the case of azacitidine, this is particularly problematic in view of the drug's cytotoxicity.

In the case of an injectable suspension of an active ingredient, it is also important that the size of the suspended particles is controlled so that they can be injected through a needle. If large, aggregated particles are present, they will not only block the needle, through which the suspension is to be injected, but also will not form a stable suspension within (i.e. they will instead tend to sink to the bottom of) the injection liquid.

There is, thus, an unmet clinical need in the treatment of conditions like MDS for a longer lasting, more effective and/or improved drug delivery system comprising azacitidine.

Atomic layer deposition (ALD) is a technique that is employed to deposit thin films comprising a variety of materials, including organic, biological, polymeric and, especially, inorganic materials, such as metal oxides, on solid substrates. It is an enabling technique for atomic and close-to-atomic scale manufacturing (ACSM) of materials, structures, devices and systems in versatile applications (see, for example, Zhang et al, Nanomanuf. Metrol. 2022, https://doi.org/10.1007/s41871-022-00136-8). Based on its self-limiting characteristics, ALD can achieve atomic-level thickness that is only controlled by adjusting the number of growth cycles. Moreover, multilayers can be deposited, and the properties of each layer can be customized at the atomic level.

Due to its atomic-level control, ALD is used as a key technique for the manufacturing of, for example, next-generation semiconductors, or in atomic-level synthesis of advanced catalysts as well as in the precise fabrication of nanostructures, nanoclusters, and single atoms (see, for example, Zhang et al. vide supra).

The technique is usually performed at low pressures and elevated temperatures. Film coatings are produced by alternating exposure of solid substrates within an ALD reactor chamber to vaporized reactants in the gas phase. Substrates can be silicon wafers, granular materials or small particles (e.g. microparticles or nanoparticles).

The coated substrate is protected from chemical reactions (decomposition) and physical changes by the solid coating. ALD can also potentially be used to control the rate of release of the substrate material within a solvent, which makes it of potential use in the formulation of active pharmaceutical ingredients.

In ALD, a first precursor, which can be metal-containing, is fed into an ALD reactor chamber (in a so called ‘precursor pulse’), and forms an adsorbed atomic or molecular monolayer at the surface of the substrate. Excess first precursor is then purged from the reactor, and then a second precursor, such as water, is pulsed into the reactor. This reacts with the first precursor, resulting in the formation of a monolayer of e.g. metal oxide on the substrate surface. A subsequent purging pulse is followed by a further pulse of the first precursor, and thus the start of a new cycle of the same events (a so called ‘ALD cycle’).

The thickness of the film coating is controlled by inter alia the number of ALD cycles that are conducted.

In a normal ALD process, because only atomic or molecular monolayers are produced during any one cycle, no discernible physical interface is formed between these monolayers, which essentially become a continuum at the surface of the substrate.

In international patent application WO 2014/187995, a process is described in which a number of ALD cycles are performed, which is followed by periodically removing the resultant coated substrates from the reactor and conducting a re-dispersion/agitation step to present new surfaces available for precursor adsorption.

The agitation step is done primarily to solve a problem observed for nano- and microparticles, namely that, during the ALD coating process, aggregation of particles takes place, resulting in ‘pinholes’ being formed by contact points between such particles. The re-dispersion/agitation step was performed by placing the coated substrates in water and sonicating, which resulted in deagglomeration, and the breaking up of contact points between individual particles of coated active substance.

The particles were then loaded back into the reactor and the steps of ALD coating of the powder, and deagglomerating the powder were repeated 3 times, to a total of 4 series of cycles. This process has been found to allow for the formation of coated particles that are, to a large extent, free of pinholes (see also, Hellrup et al., Int. J. Pharm., 529, 116 (2017)).

We have made a novel, injectable azacitidine composition, in which ALD is used to coat azacitidine microparticles with a specific mixed oxide coating layers, which coated particles are suspended in an aqueous vehicle. This composition produces an advantageous pharmacokinetic profile by releasing active ingredient over an extended period of time to provide a therapeutically-effective level of drug in systemic circulation, without any significant initial burst effect.

### DISCLOSURE OF THE INVENTION

According to a first aspect of the invention there is provided a pharmaceutical formulation that is useful in the treatment of MDS, comprising a plurality of particles suspended in an aqueous carrier system, which particles:
(a) have a weight-, number-, or volume-based mean diameter that is between amount 10 nm and about 700 μm; and
(b) comprise solid cores comprising azacitidine, or a pharmaceutically-acceptable salt thereof, coated, at least in part, by a coating of inorganic material comprising mixture of:
   (i) zinc oxide (ZnO); and
   (ii) one or more other metal and/or metalloid oxides,
   wherein the atomic ratio ((i):(ii)) is at least about 1:6 and up to and including about 6:1.
      which formulations are hereinafter referred to as ‘the formulations of the invention’.

In a preferred that the atomic ratio ((i):(ii)) is at least about 1:1 and up to and including about 6:1.

The coating comprising a mixture of zinc oxide and one or more other metal and/or metalloid oxides is referred to hereinafter as the ‘mixed oxide’ coating or coating material(s).

The term ‘solid’ will be well understood by those skilled in the art to include any form of matter that retains its shape and density when not confined, and/or in which molecules are generally compressed as tightly as the repulsive forces among them will allow. The solid cores have at least a solid exterior surface onto which a layer of coating material can be deposited. The interior of the solid cores may be also solid or may instead be hollow. For example, if the particles are spray dried before they are placed into the reactor vessel, they may be hollow due to the spray drying technique.

The solid cores of the formulation of the invention comprise azacitidine or pharmaceutically-acceptable salt thereof and, in this respect, may consist essentially of azacitidine or said salt thereof, or may include azacitidine or said salt thereof along with other excipients or other active ingredients.

By ‘consists essentially’ of azacitidine or pharmaceutically-acceptable salt thereof, we include that the solid core is essentially comprised only of azacitidine or salt thereof, i.e. it is free from non-biologically active substances, such as excipients, carriers and the like (vide infra), and from other active substances. This means that the core may comprise less than about 5%, such as less than about 3%, including less than about 2%, e.g. less than about 1% of such other excipients and/or active substances.

In the alternative, cores comprising azacitidine or pharmaceutically-acceptable salt thereof may include that active ingredient is in admixture with one or more pharmaceutical ingredients, which may include pharmaceutically-acceptable excipients, such as adjuvants, diluents or carriers, and/or may include other biologically-active ingredients.

Non-biologically active adjuvants, diluents and carriers that may be employed in cores to be coated in accordance with the invention may include pharmaceutically-acceptable substances that are soluble in water, such as carbohydrates, e.g. sugars, such as lactose and/or trehalose, and sugar alcohols, such as mannitol, sorbitol and xylitol, or pharmaceutically-acceptable inorganic salts, such as sodium chloride. Preferred carrier/excipient materials include sugars and sugar alcohols.

Azacitidine or pharmaceutically-acceptable salt thereof may be presented in a crystalline, a part-crystalline and/or an amorphous state. Azacitidine or pharmaceutically-acceptable salt thereof may be in the solid state, or may be converted into the solid state, at about room temperature (e.g. about 18° C.) and about atmospheric pressure, irrespective of the physical form. Active agent (and optionally other pharmaceutical ingredients as mentioned hereinbefore) should also remain in the form of a solid whilst being coated in, for example, an ALD reactor, and also should not decompose physically or chemically to an appreciable degree (i.e. no more than about 10% w/w) whilst being coated, or after having been covered by the mixed metal oxide coating material.

Pharmaceutically acceptable salts of azacitidine include acid addition salts and base addition salts. Such salts may be formed by conventional means, for example by reaction of a free acid or a free base form of the active ingredient with one or more equivalents of an appropriate acid or base, optionally in a solvent, or in a medium in which the salt is insoluble, followed by removal of said solvent, or said medium, using standard techniques (e.g. in vacuo, by freeze-drying or by filtration). Salts may also be prepared using techniques known to those skilled in the art, such as by exchanging a counter-ion of the active ingredient in the form of a salt with another counter-ion, for example using a suitable ion exchange resin.

Particular salts that may be mentioned include acid additional salts of, for example, hydrochloric acid, L-lactic acid, acetic acid, phosphoric acid, (+)-L-tartaric acid, citric acid, propionic acid, butyric acid, hexanoic acid, L-aspartic acid, L-glutamic acid, succinic acid, ethylenediaminetetraacetic acid (EDTA), maleic acid, methanesulfonic acid and the like.

Formulations of the invention comprise a pharmacologically-effective amount of azacitidine or pharmaceutically-acceptable salt thereof. Preferably, the solid cores of the formulation of the invention comprise said pharmacologically-effective amount of azacitidine or salt thereof.

The term ‘pharmacologically-effective amount’ refers to an amount of azacitidine or salt thereof, which is capable of conferring a desired physiological change (such as a therapeutic effect) on a treated patient, whether administered alone or in combination with another active ingredient. Such a biological or medicinal response, or such an effect, in a patient may be subjective (i.e. the subject gives an indication of, or feels, an effect), and includes at least partial alleviation of the symptoms of the disease or disorder being treated, or curing or preventing said disease or disorder, or may be objective (i.e. measurable by some test or marker).

Dosages of azacitidine/salt thereof that may be administered to a patient should thus be sufficient to affect a therapeutic response over a reasonable and/or relevant timeframe. One skilled in the art will recognize that the selection of the exact dose and composition and the most appropriate delivery regimen will also be influenced by not only the pharmacological properties of the formulation, but also inter alia the route of administration, the nature and severity of the condition being treated, and the physical condition and mental acuity of the recipient, as well as the age, condition, body weight, sex and response of the patient to be treated, and the stage/severity of the disease, as well as genetic differences between patients.

Dosages of azacitidine/salt thereof may also be determined by the timing and frequency of administration. In any event, the medical practitioner, or other skilled person, will be able to determine routinely the actual dosage of azacitidine/salt thereof, which will be most suitable for an individual patient.

When injected, formulations of the invention provide a depot formulation, from which azacitidine is released over a prolonged period of time. That period of time may be at least about 3 days, such as about 5, or about 7, clays, and up to a period of about 4 weeks, such as about 3 weeks (e.g. about 2 weeks).

Suitable doses of azacitidine or pharmaceutically-acceptable salts thereof in formulations of the invention may thus provide a plasma concentration-time profile that provides an exposure (AUC, defined as, for example AUCₗₐₛₜ (the area under plasma concentration vs. time curve up to the last detectable concentration over a prolonged period of time) or, more preferably, AUC_{∞} (the area under the plasma concentration vs. time curve up to infinite time)) that provides at least the same therapeutic effect as that obtained for current, commercial subcutaneous injections and/or intravenous infusions of azacitidine that are used in clinical practice.

Formulations of the invention may be capable of providing an exposure, in terms of AUC_{∞}, for azacitidine in plasma over any one of the above-mentioned time periods that is no more than 100% of the total exposure (AUC_{∞}) obtained from the current standard of care/dosing regimen administered over seven consecutive days by injection or infusions of azacitidine, which are currently 75 mg of azacitidine (calculated as the free compound) per m² of body surface area (BSA) administered subcutaneously or intravenously on a daily basis over 7 days.

More preferably, the total exposure (e.g. AUC_{∞}) for azacitidine over any one of the above-mentioned time periods may be at least about 50% (e.g. at least about 65%), at least about 75% (e.g. at least about 80%), such as at least about 85% of the total exposure (e.g. AUC_{∞}) obtained from the current standard of care/dosing regimen administered over seven consecutive days by injection or infusions of azacitidine (where 525 mg/m² of BSA gives an average AUC_{∞} of 960±458 ng*h/mL).

This enables a dose of azacitidine or pharmaceutically-acceptable salt thereof within a formulation of the invention that provides, or is capable of providing, a daily dose (that is the mean dose released per day from the formulation after injection over any one of the above-mentioned time periods) that is in the range of is between about 10% (e.g. about 15%) and about 80% (e.g. about 70%, such as about 65%) of the daily dose administered within the current standard of care, that is daily injection or infusion treatment (each calculated as the free compound).

Total doses that may be injected into patients by way of a formulation of the invention may thus be in the range of about 200 mg (such as about 300 mg) up to about 1000 mg per m² of BSA.

As formulations of the invention provide a steady state release of azacitidine after injection, this means that the average Cₘₐₓ (the maximum concentration observed in the plasma concentration vs. time curve) will be less than that obtained from the current standard of care/dosing regimen administered over seven consecutive days by injection or infusions of azacitidine (750±403 ng/mL). For formulations of the invention, the average Cₘₐₓ may be between about 200 and about 700 ng/mL.

The solid azacitidine-containing cores of the formulation of the invention are provided in the form of nanoparticles or, more preferably, microparticles. Preferred weight-, number-, or volume-based mean diameters are between about 50 nm (e.g. about 100 nm, such as about 250 nm) and about 30 μm, for example between about 500 nm and about 100 μm, more particularly between about 1 μm and about 50 μm, such as about 25 μm, e.g. about 20 μm.

As used herein, the term ‘weight based mean diameter’ will be understood by the skilled person to include that the average particle size is characterised and defined from a particle size distribution by weight, i.e. a distribution where the existing fraction (relative amount) in each size class is defined as the weight fraction, as obtained by e.g. sieving (e.g. wet sieving). As used herein, the term ‘number based mean diameter’ will be understood by the skilled person to include that the average particle size is characterised and defined from a particle size distribution by number, i.e. a distribution where the existing fraction (relative amount) in each size class is defined as the number fraction, as measured by e.g. microscopy. As used herein, the term ‘volume based mean diameter’ will be understood by the skilled person to include that the average particle size is characterised and defined from a particle size distribution by volume, i.e. a distribution where the existing fraction (relative amount) in each size class is defined as the volume fraction, as measured by e.g. laser diffraction. The person skilled in the art will also understand there are other suitable ways of expressing mean diameters, such as area based mean diameters, and that these other expressions of mean diameter are interchangeable with those used herein. Other instruments that are well known in the field may be employed to measure particle size, such as equipment sold by e.g. Malvern Instruments, Ltd (Worcestershire, UK) and Shimadzu (Kyoto, Japan).

Particles may be spherical, that is they possess an aspect ratio smaller than about 20, more preferably less than about 10, such as less than about 4, and especially less than about 2, and/or may possess a variation in radii (measured from the centre of gravity to the particle surface) in at least about 90% of the particles that is no more than about 50% of the average value, such as no more than about 30% of that value, for example no more than about 20% of that value.

Nevertheless, the coating of particles on any shape is also possible in accordance with the invention. For example, irregular shaped (e.g. ‘raisin’-shaped), needle-shaped, flake-shaped or cuboid-shaped particles may be coated. For a non-spherical particle, the size may be indicated as the size of a corresponding spherical particle of e.g. the same weight, volume or surface area. Hollow particles, as well as particles having pores, crevices etc., such as fibrous or ‘tangled’ particles may also be coated in accordance with the invention.

Particles may be obtained in a form in which they are suitable to be coated or be obtained in that form, for example by particle size reduction processes (e.g. crushing, cutting, milling or grinding) to a specified weight based mean diameter (as hereinbefore defined), for example by wet grinding, dry grinding, air jet-milling (including cryogenic micronization), ball milling, such as planetary ball milling, as well as making use of end-runner mills, roller mills, vibration mills, hammer mills, roller mill, fluid energy mills, pin mills, etc. Alternatively, particles may be prepared directly to a suitable size and shape, for example by spray-drying, freeze-drying, spray-freeze-drying, vacuum-drying, precipitation, including the use of supercritical fluids or other top-down methods (i.e. reducing the size of large particles, by e.g. grinding, etc.), or bottom-up methods (i.e. increasing the size of small particles, by e.g. sol-gel techniques, crystallization, etc.). Nanoparticles may alternatively be made by well-known techniques, such as gas condensation, attrition, chemical precipitation, ion implantation, pyrolysis, hydrothermal synthesis, etc.

It may be necessary (depending upon how the particles that comprise the cores are initially provided) to wash and/or clean them to remove impurities that may derive from their production, and then dry them. Drying may be carried out by way of numerous techniques known to those skilled in the art, including evaporation, spray-drying, vacuum drying, freeze drying, fluidized bed drying, microwave drying, IR radiation, drum drying, etc. If dried, cores may then be deagglomerated by grinding, screening, milling and/or dry sonication. Alternatively, cores may be treated to remove any volatile materials that may be absorbed onto its surface, e.g. by exposing the particle to vacuum and/or elevated temperature.

Surfaces of cores may be chemically activated prior to applying the first layer of coating material, e.g. by treatment with hydrogen peroxide, ozone, free radical-containing reactants or by applying a plasma treatment, in order to create free oxygen radicals at the surface of the core. This in turn may produce favourable adsorption/nucleation sites on the cores for the ALD precursors.

The azacitidine-containing cores are coated with a coating material that comprises a mixture of zinc oxide, and one or more other metal and/or metalloid oxides, at a atomic ratio of zinc oxide to the other oxide(s) that is at least about 1:6 (e.g. at least about 1:4, such as at least about 1:2), preferably at least about 1:1 (e.g. at least about 1.5:1, such as at least about 2:1), including at least about 2.25:1, such as at least about 2.5:1 (e.g. at least about 3.25:1 or least about 2.75:1 (including 3:1)), and is up to (i.e. no more than) and including about 6:1, including Lip to about 5.5:1, or up to about 5:1, such as Lip to about 4.5:1, including up to about 4:1 (e.g. up to about 3.75:1).

Preferred methods of applying the coating(s) to the cores comprising biologically-active agents include gas phase techniques, such as ALD or related technologies, such as atomic layer epitaxy (ALE), molecular layer deposition (MLD; a similar technique to ALD with the difference that molecules (commonly organic molecules) are deposited in each pulse instead of atoms), molecular layer epitaxy (MLE), chemical vapor deposition (CVD), atomic layer CVD, molecular layer CVD, physical vapor deposition (PVD), sputtering PVD, reactive sputtering PVD, evaporation PVD and binary reaction sequence chemistry. ALD is the preferred method of coating according to the invention.

When ALD is employed, the above-described mixed oxide coating may be prepared by feeding a first, zinc-, other metal- or metalloid-containing precursor into an ALD reactor chamber (in a so called ‘precursor pulse’) to form the adsorbed atomic or molecular zinc-, other metal- or metalloid-containing monolayer at the surface of the particle. A second precursor (e.g. water) is then pulsed into the reactor and reacts with the first precursor, resulting in the formation of a monolayer of zinc, metal or metalloid oxide, respectively, on the substrate surface. A subsequent purging pulse is followed by a further pulse of the first precursor, and thus the start of a new cycle of the same events, which is an ALD cycle.

In most instances, the first of the consecutive reactions will involve some functional group or free electron pairs or radicals at the surface to be coated, such as a hydroxy group (—OH) or a primary or secondary amino group (—NH₂ or —NHR where R e.g. is an aliphatic group, such as an alkyl group). The individual reactions are advantageously carried out separately and under conditions such that all excess reagents and reaction products are essentially removed before conducting the subsequent reaction.

In order to make a mixed oxide coating with an atomic ratio of (for example) between at least about 1:1 and up to and including about 6:1 of zinc oxide relative to the one or more other metal and/or metalloid oxides, the skilled person will appreciate that for every one ALD cycle (i.e. monolayer) of the other oxide(s), between about 1 and about 6 ALD cycles of zinc oxide must also be deposited. For example, for a 3:1 atomic (zinc:other oxide) mixed oxide coating to be formed, 3 zinc-containing precursor pulses may each be followed by second precursor pulses, forming 3 monolayers of zinc oxide, which will then be followed by 1 pulse of the other metal and/or metalloid-containing precursor followed by second precursor pulse, forming 1 monolayer of oxide of the other metal and/or metalloid. Alternatively, 6 monolayers of zinc oxide may be followed by 2 monolayers of the other oxide, or any other combination so as to provide an overall atomic ratio of about 3:1. In this respect, the order of pulses to produce the relevant oxides is not critical, provided that the resultant atomic ratio is in the relevant range in the end.

Metal and/or metalloid elements other than zinc that may be mentioned include alkali metals, alkaline earth metals, noble metals, transition metals, post-transition metals, lanthanoids, etc. Metal and/or metalloids that may be mentioned include aluminium, titanium, magnesium, iron, gallium, zirconium, niobium, hafnium, tantalum, lanthanum, and/or silicon; more preferably aluminium, titanium, magnesium, iron, gallium, and/or zirconium. Particular metal and/or metalloid elements that may be mentioned include aluminium and silicon.

In this respect the mixed oxide coating material preferably comprises one or other or both of aluminium oxide (Al₂O₃) and/or silicon dioxide (SiO₂).

There is provided a method of preparing of plurality of coated particles in accordance with the invention, wherein the coated particles are made by applying precursors of at least two metal and/or metal oxides forming a mixed oxide on the solid cores, and/or previously-coated solid cores, by a gas phase deposition technique. Precursors for forming a metal oxide or a metalloid oxide often include an oxygen precursor, such as water, oxygen, ozone and/or hydrogen peroxide; and a metal and/or metalloid compound, typically an organometal compound or an organometalloid compound.

Non-limiting examples of precursors are as follows: Precursors for zinc oxide may be water and diC₁-C₅alkylzinc, such as diethylzinc. Precursors for aluminium oxide may be water and triC₁-C₅alkylaluminium, such as trimethylaluminium. Precursors for silicon oxide (silica) may be water as the oxygen precursor and silanes, alkylsilanes, aminosilanes, and orthosilicic acid tetraethyl ester. Precursors for iron oxide includes oxygen, ozone and water as the oxygen precursor; and di C₁-C₅alkyl-iron, dicyclopropyl-iron, and FeCl₃. It will be appreciated that the person skilled in the art is aware of what precursors are suitable for the purpose as disclosed herein.

In ALD, layers of coating materials may be applied at process temperatures from about 20° C. to about 800° C., or from about 40° C. to about 20° C., e.g. from about 40° C. to about 150° C., such as from about 50° C. to about 100° C. The optimal process temperature depends on the reactivity of the precursors and/or substances (including biologically-active agents, e.g. azacitidine/salt) that are employed in the core and/or melting point of the core substance(s). It is preferred that a lower temperature, such as from about 30° C. to about 100° C. is employed. In particular, in one embodiment of the method a temperature from about 20° C. to about 80° C. is employed, such as from about 30° C. to about 70° C., such as from about 40° C. to about 60° C., such as about 50° C.

We have found that, when coatings comprising zinc oxide are applied using ALD at a lower temperature, such as from about 50° C. to about 100° C. (unlike other coating materials, such as aluminium oxide and titanium oxide, that form amorphous layers) the coating materials are largely crystalline in their nature.

Without being limited by theory, because zinc oxide is crystalline, if only zinc oxide is employed as coating material, we are of the understanding that interfaces may be formed between adjacent crystals of zinc oxide that are deposited by ALD, through which a carrier system, medium or solvent in which zinc oxide is partially soluble (e.g. an aqueous solvent system) can ingress following suspension therein. It is believed that this may give rise to dissolution that is too fast for the depot-forming composition that it is intended to make.

In addition, previous studies have shown that, when suspended in aqueous media, the relative bioavailability for azacitidine formulations coated with zinc oxide is lower than uncoated azacitidine. We believe that this lower relative bioavailability is due to degradation of the azacitidine before it can be released into systemic circulation. Penetration of water through crystalline interfaces within a zinc oxide coating as described above is thought to lead to hydrolysis of azacitidine within the interior of the coated particle.

We have now found that these problems may be alleviated by making a mixed oxide coating as described herein. In particular, by forming a mixed oxide coating as described herein, that is predominantly, but not entirely, comprised of zinc oxide, we have been able to coat active ingredients with coatings that appear to be essentially amorphous, or a composite between crystalline and amorphous material and/or in which ingress of injection vehicles such as water may be reduced. In this respect, it appears to us that the presence of the aforementioned perceived interfaces may be reduced, or avoided altogether, by employing the mixed oxide aspect of the invention, in either a heterogeneous manner (in which the other oxide is ‘filling in’ gaps formed by the interfaces), or in a homogeneous manner (in which a true composite of mixed oxide materials is formed during deposition, in a manner where the interfaces are potentially avoided in the first place). As described hereinafter, formulations of the invention demonstrate relative bioavailabilities that are comparable with the uncoated azacitidine.

The gas phase deposition reactor chamber used may optionally, and/or preferably, be a stationary gas phase deposition reactor chamber. The term ‘stationary’, in the context of gas phase deposition reactor chambers, will be understood to mean that the reactor chamber remains stationary while in use to perform a gas phase deposition technique, excluding negligible movements and/or vibrations such as those caused by associated machinery for example.

Additionally, a so-called ‘stop-flow’ process may be employed. Using a stop-flow process, once the first precursor has been fed into the reactor chamber and prior to the first precursor being purged from the reactor chamber, the first precursor may be allowed to contact the cores in the reactor chamber for a pre-determined period of time (which may considered as a soaking time). During the pre-determined period of time there is preferably a substantial absence of pumping that may result in flow of gases and/or a substantial absence of mechanical agitation of the cores.

The employment of the stop-flow process may increase coating uniformity by allowing each gas to diffuse conformally in high aspect-ratio substrates, such as powders. The benefits may be even more pronounced when using precursors with slow reactivity as more time is given for the precursor to react on the surface. This may be evident especially when depositing mixed oxide coatings according to the invention. For example, when depositing a mixed zinc oxide/aluminium oxide coating as described herein, we have found that a zinc-containing precursor, such as diethylzinc (DEZ), which has a lower reaction probability towards the surface of a substrate than, for example, aluminium containing precursors, such as trimethylaluminum (TMA).

In addition to generating coatings with good shell integrity and more controlled release profiles, the employment of such a stop-flow process may improve the ability to achieve a particular coating composition.

For example, when attempting to employ a gas phase technique to produce a coating comprising an atomic ratio of 3:1 between zinc and aluminium in the resulting shell as described above, we have found that a ratio that is much closed to 3:1 may be achieved using a stop-flow process than when depositing material using a continuous flow of precursors.

Preferably, and/or optionally, a ‘multi-pulse’ technique may also be employed to feed the first precursor, the second precursor or both precursors to the reactor chamber.

Using such a multi-pulse technique, the respective precursor may be fed into the reactor chamber as a plurality of ‘sub-pulses’, each lasting a short period of time such as 1 second up to about a minute (depending on the size and the nature of the gas phase deposition reactor), rather than as one continuous pulse. The precursor may be allowed to contact the cores in the reactor chamber for the pre-determined period of time, for example from about 1 to 500 seconds, about 2 to 250 seconds, about 3 to 100 seconds, about 4 to 50 seconds, or about 5 to 10 seconds, for example 9 seconds, after each sub-pulse. Again, depending on the size and the nature of the gas phase deposition reactor, this time could be extended up to several minutes (e.g. up to about 30 minutes). The introduction of a sub-pulse followed by a period of soaking time may be repeated a pre-determined number of times, such as between about 5 to 1000 times, about 10 to 250 times, or about 20 to 50 times in a single step.

The cores may be coated with one or more separate, discrete layers, of mixed oxide coatings as defined herein. Preferably, more than one separate, discrete mixed oxide layer, coating or shell (which terms are used herein interchangeably) is applied (that is ‘separately applied’) to the solid cores comprising azacitidine sequentially.

By ‘separate application’ of ‘separate layers, coatings or shells’, we mean that the solid cores are coated with a first layer of coating material, which layer is formed by more than one (e.g. a plurality or a set of) cycles as described herein, each cycle producing a monolayer of zinc oxide, or other metal and/or metalloid oxide (as appropriate), and then that resultant coated core is subjected to some form of deagglomeration process.

In other words, ‘gas-phase deposition (e.g. ALD) cycles’ can be repeated several times to provide a ‘gas-phase deposition (e.g. ALD) set’ of cycles, which may consist of e.g. 10, 25 or 100 cycles. However, after this set of cycles, the coated core is subjected to some form of deagglomeration process, which is followed by a further set of cycles.

This process may be repeated as many times as is desired and, accordingly, the number of discrete layers of coating material(s) produced by sets of cycles that is in a final coating corresponds to the number of these intermittent deagglomeration steps with a final mechanical deagglomeration being conducted prior to the application of a final layer (set of cycles) of coating material.

The terms ‘disaggregation’ and ‘deagglomeration’ are used interchangeably when referring to the coated particles, and disaggregating coated particles aggregates is preferably done by way of a mechanical sieving technique.

Coated cores may be subjected to the aforementioned deagglomeration process internally, without being removed from said apparatus by way of a continuous process. Such a process will involve forcing solid product mass formed by coating said cores through a sieve that is located within the reactor, and is configured to deagglomerate any particle aggregates upon forcing of the coated cores by means of a forcing means applied within said reactor, prior to being subjected to a second and/or a further coating. This process is continued for as many times as is required and/or appropriate prior to the application of the final coating as described herein.

Having the sieve located within the reactor vessel means that the coating can be applied by way of a continuous process which does not require the particles to be removed from the reactor. Thus, no manual handling of the particles is required, and no external machinery is required to deagglomerate the aggregated particles. This not only considerably reduces the time of the coating process being carried out, but is also more convenient and reduces the risk of harmful (e.g. poisonous) materials being handled by personnel. It also enhances the reproducibility of the process by limiting the manual labour and reduces the risk of contamination.

Alternatively, and/or preferably, coated cores may be removed from the coating apparatus, such as the ALD reactor, and thereafter subjected to an external deagglomeration step, for example as described in international patent application WO 2014/187995. Such an external deagglomeration step may comprise agitation, such as sonication in the wet or dry state, or preferably may comprise subjecting the resultant solid product mass that has been discharged from the reactor to sieving, e.g. by forcing it through a sieve or mesh in order to deagglomerate the particles, for example as described hereinafter, prior to placing the particles back into the coating apparatus for the next coating step. Again, this process may be continued for as many times as is required and/or appropriate prior to the application of the final coating.

In an external deagglomeration process, deagglomeration may alternatively be effected (additionally and/or instead of the abovementioned processes) by way of subjecting the coated particles in the wet or dry state to one or more of nozzle aerosol generation, milling, grinding, stirring, high sheer mixing and/or homogenization. If the step(s) of deagglomeration are carried out on particles in the wet state, the deagglomerated particles should be dried (as hereinbefore described in relation to cores) prior to the next coating step.

However, we prefer that, in such an external process, the deagglomeration step(s) comprise one or more sieving step(s), which may comprise jet sieving, manual sieving, vibratory sieve shaking, horizontal sieve shaking, tap sieving, or (preferably) sonic sifting as described hereinafter, or a like process, including any combination of these sieving steps. Manufacturers of suitable sonic sifters include Advantech Manufacturing, Endecott and Tsutsui.

Vibrational sieving techniques may involve a means of vibrationally forcing the solid product mass formed by coating said cores through a sieve that is located internally or (preferably) externally to (i.e. outside of) the reactor, and is configured to deagglomerate any particle aggregates upon said vibrational forcing of the coated cores, prior to being subjected to a second and/or a further layer of coating material. This process is repeated as many times as is required and/or appropriate prior to the application of a final layer of coating material.

Vibrational forcing means comprises a vibration motor which is coupled to a sieve. The vibration motor is configured to vibrate and/or gyrate when an electrical power is supplied to it. For example, the vibration motor may be a piezoelectric vibration motor comprising a piezoelectric material which changes shape when an electric field is applied, as a consequence of the converse piezoelectric effect. The changes in shape of the piezoelectric material cause acoustic or ultrasonic vibrations of the piezoelectric vibration motor.

The vibration motor may alternatively be an eccentric rotating mass (ERM) vibration motor comprising a mass which is rotated when electrical power is supplied to the motor. The mass is eccentric from the axis of rotation, causing the motor to be unbalanced and vibrate and/or gyrate due to the rotation of the mass. Further, the ERM vibration motor may comprise a plurality of masses positioned at different locations relative to the motor. For example, the ERM vibration motor may comprise a top mass and a bottom mass each positioned at opposite ends of the motor. By varying each mass and its angle relative to the other mass, the vibrations and/or gyrations of the ERM vibration motor can be varied.

The vibration motor is coupled to the sieve in a manner in which vibrations and/or gyrations of the motor when electrical power is supplied to it are transferred to the sieve.

The sieve and the vibration motor may be suspended from a mount (such as a frame positionable on a floor, for example) via a suspension means such that the sieve and motor are free to vibrate relative to the mount without the vibrations being substantially transferred to or dampened by the mount. This allows the vibration motor and sieve to vibrate and/or gyrate without impediment and also reduces noise generated during the vibrational sieving process. The suspension means may comprise one or more springs or bellows (i.e. air cushion or equivalent cushioning means) that couple the sieve and/or motor to the mount. Manufacturers of vibratory sieves or sifters suitable for carrying out such a process include for instance RLssell Finex, SWECO, Filtra Vibracion, VibraScreener, Gough Engineering and Farley Greene.

Preferably, the vibrational sieving technique further comprises controlling a vibration probe coupled to the sieve. The vibration probe may be controlled to cause the sieve to vibrate at a separate frequency to the frequency of vibrations caused by the vibration motor. Preferably the vibration probe causes the sieve to vibrate at a higher frequency than the vibrations caused by the vibration motor and, more preferably, the frequency is within the ultrasonic range.

Providing additional vibrations to the sieve by means of the vibration probe reduces the occurrence of clogging in the sieve, reduces the likelihood of the sieve being overloaded and decreases the amount of time needed to clean the mesh of the sieve.

Preferably, the aforesaid vibrational sieving technique comprises sieving coated particles with a throughput of at least 1 g/minute. More preferably, the vibrational sieving technique comprises sieving coated particles with a throughput of 4 g/minute or more.

The throughput depends on the area of the sieve mesh, mesh-size of the sieve, the particle size, the stickiness of the particles, static nature of the particle. By combining some of these features a much higher throughput is possible. Accordingly, the vibrational sieving technique may more preferably comprise sieving coated particles with a throughput of up to 1 kg/minute or even higher.

Any one of the above-stated throughputs represents a significant improvement over the use of known mechanical sieving, or sifting, techniques. For example, we found that sonic sifting involved sifting in periods of 15 minutes with a 15-minute cooling time in-between, which is necessary for preserving the apparatus. To sift 20 g of coated particles required 9 sets of 15 minutes of active sifting time, i.e. a total time (including the cooling) of 255 minutes. By comparison, by using the aforementioned vibrational sieving technique, 20 g of coated particles may be sieved continuously in, at most, 20 minutes, or more preferably in just 5 minutes, or less.

The sieve mesh size may be determined so that the ratio of the size of the sieved or sonic sifted particles to the sieve mesh size is about 1:>1, preferably about 1:2, and optionally about 1:4. The size mesh size may range from about 20 μm to about 100 μm, preferably from about 20 μm to about 60 μm.

Appropriate sieve meshes may include perforated plates, microplates, grid, diamond, threads, polymers or wires (woven wire sieves) but are preferably formed from metals, such as stainless steel.

Surprisingly, using a stainless steel mesh within the vibrational sieving technique is as gentle to the particle coatings as using a softer polymer sieve as part of a mechanical sieving technique such as sonic sifting.

Also, a known problem with sieving powders is the potentially dangerous generation of static electricity. A steel mesh has the advantage of removing static electricity from the powder while that is not the case with a polymeric mesh, which has to be used in a sonic sifter.

Further, the mesh size of known sonic sifters is limited to about 100 μm since the soundwaves travel through the mesh rather than vibrating it. That limitation does not exist using for vibrational sieving techniques as there is no reliance on soundwaves to generate vibrations in the sieve. Therefore, the vibrational sieving technique described herein allows larger particles to be sieved than if alternative mechanical sieving techniques were used.

If a (e.g. vibrational) sieve is located externally to (i.e. outside of) the reactor, the process for making coated cores of formulations of the invention comprises discharging the coated particles from the gas phase deposition reactor prior to subjecting the coated particles to agitation, followed by reintroducing the deagglomerated, coated particles into the gas phase deposition reactor prior to applying a further layer of at least one coating material to the reintroduced particles.

We have found that applying separate layers of coating materials following external deagglomeration gives rise to visible and discernible interfaces that may be observed by analysing coated particles according to the invention, and are observed by e.g. TEM as regions of higher electron permeability. In this respect, the thickness of the layers between interfaces correspond directly to the number of cycles in each series that are carried out within the ALD reactor, and between individual external agitation steps.

Because, in an ALD coating process, coating takes place at the atomic level, such clear, physical interfaces are typically more difficult to observe.

Without being limited by theory, it is believed that removing coated particles from the vacuum conditions of the ALD reactor and exposing a newly-coated surface to the atmosphere results in structural rearrangements due to relaxation and reconstruction of the outermost atomic layers. Such a process is believed to involve rearrangement of surface (and near surface) atoms, driven by a thermodynamic tendency to reduce surface free energy.

Furthermore, surface adsorption of species, e.g. hydrocarbons that are always present in the air, may contribute to this phenomenon, as can surface modifications, due to reaction of coatings formed with hydrocarbons, as well as atmospheric oxygen and the like. Accordingly, if such interfaces are analysed chemically, they may contain traces of contaminants or the core material, such as active ingredient that forms part of the core, that do not originate from the coating process, such as ALD.

Whether carried out inside or outside of the reactor, particle aggregates are preferably broken Lip by a forcing means that forces them through a sieve, thus separating the aggregates into individual particles or aggregates of a desired and predetermined size (and thereby achieving deagglomeration). In the latter regard, in some cases the individual primary particle size is so small (i.e. <1 μm) that achieving ‘full’ deagglomeration (i.e. where aggregates are broken down into individual particles) is not possible. Instead, deagglomeration is achieved by breaking down larger aggregates into smaller aggregates of secondary particles of a desired size, as dictated by the size of the sieve mesh. The smaller aggregates are then coated by the gas phase technique to form fully coated ‘particles’ in the form of small aggregate particles. In this way, the term ‘particles’, when referring to the particles that have been deagglomerated and coated in the context of the invention, refers to both individual (primary) particles and aggregate (secondary) particles of a desired size.

In any event, the desired particle size (whether that be of individual particles or aggregates of a desired size) is maintained and, moreover, continued application of the gas phase coating mechanism to the particles after such deagglomeration via the sieving means that a complete coating is formed on the particle, thus forming fully coated particles (individual or aggregates of a desired size).

Whether carried out inside or outside of the reactor, the above-described repeated coating and deagglomeration process may be carried out at least 1, preferably 2, more preferably 3, such as 4, including 5, more particularly 6, e.g. 7 times, and no more than about 100 times, for example no more than about 50 times, such as no more than about 40 times, including no more than about 30 times, such as between 2 and times, e.g. between 3 and 15 times, such as 10 times, e.g. 9 or 8 times, more preferably 6 or 7 times, and particularly 4 or 5 times.

Whether carried out inside or outside of the reactor, it is preferred that at least one sieving step is carried out and further that that step preferably comprises a vibrational sieving step as described above. It is further preferred that at least the final sieving step comprises a vibrational sieving step being conducted prior to the application of a final layer (set of cycles) of coating material. However, it is further preferred that more than one (including each) of the sieving steps comprise vibrational sieving techniques, steps or processes as described herein.

The preferable repetition of these steps makes the improved throughput of any vibrational sieving technique all the more beneficial.

The total thickness of the coating (meaning all the separate layers/coatings/shells) will on average be in the region of between about 0.5 nm and about 2 μm.

The minimum thickness of each individual layer/coating/shell will on average be in the region of about 0.1 nm (including about 0.5 nm, for example about 0.75 nm, such as about 1 nm).

The maximum thickness of each individual layer/coating/shell will depend on the size of the core (to begin with), and thereafter the size of the core with the coatings that have previously been applied, and may be on average about 1 hundredth of the mean diameter (i.e. the weight-, number-, or volume-based mean diameter) of that core, or core with previously-applied coatings.

Preferably, for particles with a mean diameter that is between about 100 nm and about 1 μm, the total coating thickness should be on average between about 1 nm and about nm; for particles with a mean diameter that is between about 1 μm and about 20 μm, the coating thickness should be on average between about 1 nm and about 10 nm; for particles with a mean diameter that is between about 20 μm and about 700 μm, the coating thickness should be on average between about 1 nm and about 100 nm.

We have found that applying coatings/shells followed by conducting one or more deagglomeration step such as sonication gives rise to abrasions, pinholes, breaks, gaps, cracks and/or voids (hereinafter ‘cracks’) in the layers/coatings, due to coated particles essentially being more tightly ‘bonded’ or ‘glued’ together directly after the application of a thicker coating. This may expose a core comprising biologically-active ingredient (i.e. azacitidine/salt) to the elements once deagglomeration takes place.

As it is intended to provide particles in an aqueous suspension prior to administration to a patient, it is necessary to provide deagglomerated primary particles without pinholes or cracks in the coatings. Such cracks will result in an undesirable initial peak (burst) in plasma concentration of active ingredient directly after administration.

We have found that, by conducting one or more of the deagglomeration steps described herein, this gives rise to significantly less pinholes, gaps or cracks in the final layer of coating material, giving rise to particles that are not only completely covered by that layer/coating, but are also covered in a manner that enables the particles to be deagglomerated readily (e.g. using a non-aggressive technique, such as vortexing) in a manner that does not destroy the layers of coating material that have been formed, prior to, and/or during, pharmaceutical formulation.

In this respect, the mixed oxide coating typically completely surrounds, encloses and/or encapsulates said solid cores comprising active ingredient(s). In this way, the risk of an initial drug concentration burst due to the drug coming into direct contact with solvents in which the relevant active ingredient is soluble is minimized. This may include not only bodily fluids, but also any medium in which such coated particles may be suspended prior to injection.

Thus in a further embodiment of the invention, there are provided particles as hereinbefore disclosed, wherein said coating surrounding, enclosing and/or encapsulating said core covers at least about 50%, such as at least about 65%, including at least about 75%, such as at least about 80%, more particularly at least about 90%, such as at least about 91%, such as at least about 92%, such as at least about 93%, such as at least about 94%, such as at least about 95%, such as at least about 96%, such as at least about 97%, such as at least about 98%, such as at least about 99%, such as approximately, or about, 100%, of the surface of the solid core, such that the coating essentially completely surrounds, encloses and/or encapsulates said core.

As used herein, the term ‘essentially completely coating completely surrounds, encloses and/or encapsulates said core’ means a covering of at least about 98%, or at least about 99%, of the surface of the solid core.

In the alternative, processes described herein may result in the deagglomerated coated particles with the essential absence of said cracks through which active ingredient can be released in an uncontrolled way.

Although some minor cracks may appear in the said coating without effecting the essential function thereof in terms of controlling release, in a further embodiment, there are provided particles as hereinbefore disclosed, wherein at least about 90% of the particles do not exhibit cracks in the coating surrounding, enclosing and/or encapsulating said core. In one embodiment at least about 91%, such as at least about 92%, such as at least about 93%, such as at least about 94%, such as at least about 95%, such as at least about 96%, such as at least about 97%, such as at least about 98%, such as at least about 99%, such as approximately 100% of the particles do not exhibit said cracks.

Alternatively, by ‘essentially free of said cracks’ in the coating(s), we also mean that less than about 1% of the surfaces of the coated particles comprise abrasions, pinholes, breaks, gaps, cracks and/or voids through which active ingredient is potentially exposed (to, for example, the elements).

The layers of coating material may, taken together, be of an essentially uniform thickness over the surface area of the particles. By ‘essentially uniform’ thickness, we mean that the degree of variation in the thickness of the coating of at least about 10%, such as about 25%, e.g. about 50%, of the coated particles that are present in a formulation of the invention, as measured by TEM, is no more than about ±20%, including ±50% of the average thickness.

In addition to the essential mixed oxide coating that is employed in formulations of the invention, other coating materials, which may be pharmaceutically-acceptable and essentially non-toxic coating materials may also be applied in addition, either between separate mixed oxide coatings (e.g. in-between separate deagglomeration steps) and/or whilst a mixed oxide coating is being applied herein. Such materials may comprise multiple layers or composites of said mixed oxide and one or more different inorganic or organic materials, to modify the properties of the layer(s).

Additional coating materials may comprise organic or polymeric materials, such as a polyamide, a polyimide, a polyurea, a polyurethane, a polythiourea, a polyester or a polyimine. Additional coating materials may also comprise hybrid materials (as between organic and inorganic materials), including materials that are a combination between a metal, or another element, and an alcohol, a carboxylic acid, an amine or a nitrile. However, we prefer that coating materials comprise inorganic materials.

Additional inorganic coating materials may comprise other compounds of metals and/or metalloids, such as oxides, nitrides, sulphides, selenides, carbonates, other ternary compounds, etc. Metal, and metalloid, hydroxides and, especially, oxides are preferred, especially metal oxides.

In addition, oxides of elements other than zinc, aluminium or silicon that may be mentioned include alkali metals, alkaline earth metals, noble metals, transition metals, post-transition metals, lanthanoids, etc. Metal and metalloids that may be mentioned include titanium, magnesium, iron, gallium, zirconium, niobium, hafnium, tantalum and/or lanthanum; more preferably titanium, magnesium, iron, gallium and/or zirconium.

Additional coating materials that may be mentioned, thus, include those comprising titanium dioxide (TiO₂), iron oxides (FeₓO_{y}, e.g. FeO and/or Fe₂O₃ and/or Fe₃O₄), gallium oxide (Ga₂O₃), magnesium oxide (MgO), niobium oxide (Nb₂O₅), hafnium oxide (HfO₂), tantalum oxide (Ta₂O₅), lanthanum oxide (La₂O₃) and/or zirconium dioxide (ZrO₂).

Although the plurality of mixed oxide coated particles in accordance with the invention are essentially free of the aforementioned cracks in the applied coatings, through which active ingredient is potentially exposed (to, for example, the elements), two further, optional steps may be applied to the plurality of coated particles prior to subjecting it to further pharmaceutical formulation processing.

The first optional step may comprise, subsequent to the final deagglomeration step as hereinbefore described, application of a final overcoating layer, the thickness of which outer ‘overcoating’ layer/coating, or ‘sealing shell’ (which terms are used herein interchangeably), must be thinner than the previously-applied separate layers/coatings/shells (or ‘subshells’).

The thickness may therefore be on average no more than a factor of about 0.7 (e.g. about 0.6) of the thickness of the widest previously-applied subshell. Alternatively, the thickness may be on average no more than a factor of about 0.7 (e.g. about 0.6) of the thickness of the last subshell that is applied, and/or may be on average no more than a factor of about 0.7 (e.g. about 0.6) of the average thickness of all of the previously-applied subshells. The thickness may be on average in the region of about 0.3 nm to about 10 nm, for particles up to about 20 μm. For larger particles, the thickness may be on average no more than about 1/1000 of the coated particles' weight-, number-, or volume-based mean diameter.

The role of such as sealing shell is to provide a ‘sealing’ overcoating layer on the particles, covering over those cracks, so giving rise to particles that are not only completely covered by that sealing shell, but also covered in a manner that enables the particles to be deagglomerated readily (e.g. using a non-aggressive technique, such as vortexing) in a manner that does not destroy the subshells that have been formed underneath, prior to, and/or during, pharmaceutical formulation.

For the reasons described herein, it is preferred that the sealing shell does not comprise zinc oxide. The sealing shell may on the other hand comprise silicon dioxide or, more preferably, aluminium oxide.

The second optional step may comprise ensuring that the few remaining particles with broken and/or cracked shells/coatings are subjected to a treatment in which all particles are suspended in a solvent in which the azacitidine or salt thereof is soluble (e.g. with a solubility of at least about 0.1 mg/mL), but the least soluble material in the mixed oxide coating is insoluble (e.g. with a solubility of no more than about 0.1 μg/mL), followed by separating solid matter particles from solvent by, for example, centrifugation, sedimentation, flocculation and/or filtration, resulting in mainly intact particles being left.

The above-mentioned optional step provides a means of potentially reducing further the likelihood of a (possibly) undesirable initial peak (burst) in plasma concentration of active ingredient, as discussed hereinbefore.

At the end of the process, coated particles may be dried using one or more of the techniques that are described hereinbefore for drying cores. Drying may take place in the absence, or in the presence, of one or more pharmaceutically-acceptable excipients (e.g. a sugar or a sugar alcohol).

Alternatively, at the end of the process, separated particles may be resuspended in a solvent (e.g. water, with or without the presence of one or more pharmaceutically acceptable excipients as defined herein), for subsequent storage and/or administration to patients.

Prior to applying the first layer of coating material or between successive coatings, cores and/or partially coated particles may be subjected to one or more alternative and/or preparatory surface treatments. In this respect, one or more intermediary layers comprising different materials (i.e. other than the inorganic material(s)) may be applied to the relevant surface, e.g. to protect the cores or partially-coated particles from unwanted reactions with precursors during the coating step(s)/deposition treatment, to enhance coating efficiency, or to reduce agglomeration.

An intermediary layer may, for example, comprise one or more surfactants, with a view to reducing agglomeration of particles to be coated and to provide a hydrophilic surface suitable for subsequent coatings. Suitable surfactants in this regard include well known non-ionic, anionic, cationic or zwitterionic surfactants, such as the Tween series, e.g. Tween 80. Alternatively, cores may be subjected to a preparatory surface treatment if the active ingredient that is employed as part of (or as) that core is susceptible to reaction with one or more precursor compounds that may be present in the gas phase during the coating (e.g. the ALD) process.

Application of ‘intermediary’ layers/surface treatments of this nature may alternatively be achieved by way of a liquid phase non-coating technique, followed by a lyophilisation, spray drying or other drying method, to provide particles with surface layers to which coating materials may be subsequently applied.

Outer surfaces of particles of formulations of the invention may also be derivatized or functionalized, e.g. by attachment of one or more chemical compounds or moieties to the outer surfaces of the final layer of coating material, e.g. with a compound or moiety that enhances the targeted delivery of the particles within a patient to whom the nanoparticles are administered. Such a compound may be an organic molecule (such as PEG) polymer, an antibody or antibody fragment, or a receptor-binding protein or peptide, etc.

Alternatively, the moiety may be an anchoring group such as a moiety comprising a silane function (see, for example, Herrera et al., J. Mater. Chem., 18, 3650 (2008) and U.S. Pat. No. 8,097,742). Another compound, e.g. a desired targeting compound may be attached to such an anchoring group by way of covalent bonding, or non-covalent bonding, including hydrogen bonding, or van der Waals bonding, or a combination thereof.

The presence of such anchoring groups may provide a versatile tool for targeted delivery to specific sites in the body. Alternatively, the use of compounds such as PEG may cause particles to circulate for a longer duration in the blood stream, ensuring that they do not become accumulated in the liver or the spleen (the natural mechanism by which the body eliminates particles, which may prevent delivery to diseased tissue).

Cores coated with a mixed oxide coating, whether in the form of separate, discrete layers, coatings or shells or otherwise, as defined herein are referred to hereinafter as ‘the coated particles of the formulation of the invention’.

Pharmaceutical (or veterinary) formulations of the invention may include particles of different types, for example particles comprising different functionalization (as described hereinbefore), particles of different sizes, and/or different thicknesses of the layers of mixed oxide coating materials, or a combination thereof. By combining, in a single pharmaceutical formulation, particles with different coating thicknesses and/or different core sizes, the drug release following administration to patient may be controlled (e.g. varied or extended) over a specific time period.

Formulations of the invention may be administered systemically, for example by injection or infusion, intravenously or intraarterially (including by intravascular or other perivascular devices/dosage forms (e.g. stents)), intramuscularly, intraosseously, intracerebrally, intracerebroventricularly, intrasynovially, intrasternally, intrathecally, intralesionally, intracranially, intratumorally, cutaneously, intracutaneous, subcutaneously, transdermally, in the form of a pharmaceutically- (or veterinarily) acceptable dosage form.

The preparation of formulation of the invention comprises incorporation of coated particles as described herein into an appropriate pharmaceutically-acceptable aqueous carrier system, and may be achieved with due regard to the intended route of administration and standard pharmaceutical practice. Thus, appropriate excipients should be chemically inert to the active agent that is employed, and have no detrimental side effects or toxicity under the conditions of use. Such pharmaceutically-acceptable carriers may also impart an immediate, or a modified, release of active agent (i.e. azacitidine/salt) from the particles of the formulations of the invention.

Sterile aqueous suspensions of the particles of the formulation of the invention may be formulated according to techniques known in the art. The aqueous media should contain at least about 50% water, but may also comprise other aqueous excipients, such as Ringer's solution, and may also include polar co-solvents (e.g. ethanol, glycerol, propylene glycol, 1,3-butanediol, polyethylene glycols of various molecular weights and tetraglycol); viscosity-increasing, or thickening, agents (e.g. carboxymethylcellulose, microcrystalline cellulose, hydroxypropylmethyl cellulose, hydroxyethyl cellulose, ethyl hydroxyethyl cellulose, sodium starch glycolate, Poloxamers, such as Poloxamer 407, polyvinylpyrrolidone, cyclodextrins, such as hydroxypropyl-β-cyclodextrin, polyvinylpyrrolidone and polyethylene glycols of various molecular weights); surfactant/wetting agents to achieve a homogenous suspension (e.g. sorbitan esters, sodium lauryl sulfate; monoglycerides, polyoxyethylene esters, polyoxyethylene alkyl ethers, polyoxylglycerides and, preferably, Tweens (Polysorbates), such as Tween 80 and Tween 20). Preferred ingredients include isotonicity-modifying agents (e.g. sodium lactate, dextrose and, especially, sodium chloride); pH adjusting and/or buffering agents (e.g. citric acid, sodium citrate, and especially phosphate buffers, such as disodium hydrogen phosphate dihydrate, sodium acid phosphate, sodium dihydrogen phosphate monohydrate, and combinations thereof, which may be employed in combination with standard inorganic acids and bases, such as hydrochloric acid and sodium hydroxide); as well as other ingredients, such as mannitol, croscarmellose sodium and hyaluronic acid.

Formulations of the invention may further be formulated in the form of injectable suspension of coated particles with a size distribution that is both even and capable of forming a stable suspension within the injection liquid (i.e. without settling) and may be injected through a needle. In this respect, the formulations of the invention may comprise an aqueous medium that comprises inactive ingredients that may prevent premature gelling of the formulations of the invention, and or is viscous enough to prevent sedimentation, leading to suspensions that are not ‘homogeneous’ and thus the risk of under or overdosing of active ingredient.

Formulations may thus be stored under normal storage conditions, and maintain their physical and/or chemical integrity. The phrase ‘maintaining physical and chemical integrity’ essentially means chemical stability and physical stability.

By ‘chemical stability’, we include that any formulation of the invention may be stored (with or without appropriate pharmaceutical packaging), under normal storage conditions, with an insignificant degree of chemical degradation or decomposition.

By ‘physical stability’, we include that the any formulation of the invention may be stored (with or without appropriate pharmaceutical packaging), under normal storage conditions, with an insignificant degree of physical transformation, such as sedimentation as described above, or changes in the nature and/or integrity of the coated particles, for example in the coating itself or the active ingredient (including dissolution, solvatisation, solid state phase transition, etc.).

Examples of ‘normal storage conditions’ for formulations of the invention include temperatures of between about −50° C. and about +80° C. (preferably between about −25° C. and about +75° C., such as about 50° C.), and/or pressures of between about 0.1 and about 2 bars (preferably atmospheric pressure), and/or exposure to about 460 lux of UV/visible light, and/or relative humidities of between about 5 and about 95% (preferably about 10 to about 40%), for prolonged periods (i.e. greater than or equal to about twelve, such as about six months).

Under such conditions, formulations of the invention may be found to be less than about 15%, more preferably less than about 10%, and especially less than about 5%, chemically and/or physically degraded/decomposed, as appropriate. The skilled person will appreciate that the above-mentioned upper and lower limits for temperature and pressure represent extremes of normal storage conditions, and that certain combinations of these extremes will not be experienced during normal storage (e.g. a temperature of 50° C. and a pressure of 0.1 bar).

Formulations of the invention may comprise between about 1% to about 99%, such as between about 10% (such as about 20%, e.g. about 50%) to about 90% by weight of the coated particles with the remainder made up by carrier system and/or other pharmaceutically-acceptable excipients.

Formulations of the invention may be in the form of a liquid, a sol or a gel, which is administrable via a surgical administration apparatus, e.g. a needle, a catheter or the like, to form a depot formulation.

In any event, the preparation of suitable formulations may be achieved non-inventively by the skilled person using routine techniques. Formulations of the invention and dosage forms comprising them, may thus be formulated with conventional pharmaceutical additives and/or excipients used in the art for the preparation of pharmaceutical formulations, and thereafter incorporated into various kinds of pharmaceutical preparations and/or dosage forms using standard techniques (see, for example, Lachman et al., ‘The Theory and Practice of Industrial Pharmacy’, Lea & Febiger, 3^{rd} edition (1986); ‘Remington: The Science and Practice of Pharmacy’, Troy (ed.), University of the Sciences in Philadelphia, 21^{st} edition (2006); and/or ‘Auiton's Pharmaceutics: The Design and Manufacture of Medicines’, Aulton and Taylor (eds.), Elsevier, 4^{th} edition, 2013), and the documents referred to therein, the relevant disclosures in all of which documents are hereby incorporated by reference.

According to a further aspect of the invention there is provided a process for the preparation of a formulation of the invention which comprises mixing together the coated particles as described herein with the aqueous carrier system, for example as described herein.

For subcutaneous and/or intramuscular injections, the formulations of the invention may be presented in the form of sterile injectable and/or infusible dosage forms administrable via a surgical administration apparatus (e.g. a syringe with a needle for injection, a catheter or the like), to form a depot formulation.

There is further provided an injectable and/or infusible dosage form comprising a formulation of the invention, wherein said formulation is contained within a reservoir that is connected to, and/or is associated with, an injection or infusion means (e.g. a syringe with a needle for injection, a catheter or the like).

Alternatively, formulations of the invention can be stored prior to being loaded into a suitable injectable and/or infusible dosing means (e.g. a syringe with a needle for injection), or may even be prepared immediately prior to loading into such a dosing means.

Sterile injectable and/or infusible dosage forms may thus comprise a receptacle or a reservoir in communication with an injection or infusion means into which a formulation of the invention may be pre-loaded, or may be loaded at a point prior to use, or may comprise one or more reservoirs, within which coated particles of the formulation of the invention and the aqueous carrier system are housed separately, and in which admixing occurs prior to and/or during injection or infusion.

There is thus further provided a kit of parts comprising:
(a) coated particles of the formulation of the invention; and
(b) a carrier system of the formulation of the invention,
   as well as a kit of parts comprising coated particles of the formulation of the invention along with instructions to the end user to admix those particles with a carrier system according to the invention.

There is further provided a pre-loaded injectable and/or infusible dosage form as described herein above, but modified by comprising at least two chambers, within one of which chamber is located the coated particles of the formulation of the invention and within the other of which is located the aqueous carrier system of the formulation of the invention, wherein admixing, giving rise to a suspension or otherwise, occurs prior to and/or during injection or infusion.

Formulations of the invention may be used in human medicine. Formulations of the invention are particularly useful in any indication in which azacitidine is either approved for use in, or otherwise known to be useful in. In particular, formulations of the invention are useful in the treatment of cancers, such as MDS.

The term ‘MDS’ will be understood to include any cancerous condition characterised by non-maturation of immature blood cells in the bone marrow, meaning that they do not become healthy blood cells, which may be manifest symptomatically initially by feelings of fatigue, shortness of breath, bleeding, anaemia, frequent infections, as well as a combination of one or more of a decrease red blood cell count, decreased platelet count and/or decreased white blood cell count, and/or an increased in percentage of blasts in the bone marrow or blood.

The term ‘MDS’ also includes different sub-types, which may be determined routinely by the physician by evaluation of the aforementioned blood cell, platelet and/or blast count. The term thus includes refractory anaemia (RA), refractory anaemia with ringed sideroblasts (RARS), refractory cytopenia with multilineage dysplasia (RCMD), refractory cytopenia with multilineage dysplasia and ringed sideroblasts (RCMD-RS), refractory anemia with excess blasts (RAEB), refractory anaemia with excess blasts in transformation (RAEB-T), unclassified MDS (MDS-U) and MDS associated with isolated del (5q).

In the context of the present invention, MDS may also be classified as disease that has developed and/or is in the process of developing into leukaemia, such as acute myeloid leukaemia (AML), as well as chronic myelomonocytic leukaemia (CMML) and juvenile myelomonocytic leukaemia (JMML), both of which may be classified as mixed myelodysplastic/myeloproliferative diseases.

Formulations of the invention are indicated in the therapeutic, palliative, and/or diagnostic treatment, as well as the prophylactic treatment (by which we include preventing and/or abrogating deterioration and/or worsening of a condition) of any of the above conditions.

In the treatment of any of the above conditions, azacitidine may be combined with other treatments that are known to be useful in the treatment of the relevant conditions. This includes known anticancer drugs, and particularly those that are known to be useful in the treatment of MDS, AML, CMML and/or JMML, such as decitabine, cedazuridine and/or lenalidomide, as well as non-chemotherapeutic treatments, such as iron, all-trans retinoic acid, allogeneic stem cell transplantation and/or platelet transfusions.

In addition, as described below, we have found that injection of formulations of the invention may cause a mild inflammatory response. Such a response may be alleviated by co-administration with an antiinflammatory agent that is suitable for injection.

Appropriate antiinflammatory agents that may be employed in this regard include butylpyrazolidines (such as phenylbutazone, mofebutazone, oxyphenbutazone, clofezone, kebuzone and suxibuzone); acetic acid derivatives and related substances (indomethacin, sulindac, tolmetin, zomepirac, diclofenac, alclofenac, bumadizone, etodolac, lonazolac, fentiazac, acemetacin, difenpiramide, oxametacin, proglumetacin, ketorolac, aceclofenac and bufexamac); oxicams (such as piroxicam, tenoxicam, droxicam, lornoxicam and meloxicam); propionic acid derivatives (such as ibuprofen, naproxen, ketoprofen, fenoprofen, fenbufen, benoxaprofen, suprofen, pirprofen, flurbiprofen, indoprofen, tiaprofenic acid, oxaprozin, ibuproxam, dexibuprofen, flunoxaprofen, alminoprofen, dexketoprofen, vedaprofen, carprofen and tepoxalin); fenamates (such as mefenamic acid, tolfenamic acid, flufenamic acid, meclofenamic acid and flunixin), coxibs (such as celecoxib, rofecoxib, valdecoxib, parecoxib, etoricoxib, lumiracoxib, firocoxib, robenacoxib, mavacoxib and cimicoxib); other non-steroidal antiinflammatory agents (such as nabumetone, niflumic acid, azapropazone, glucosamine, benzydamine, glucosaminoglycan polysulfate, proquazone, orgotein, nimesulide, feprazone, diacerein, morniflumate, tenidap, oxaceprol, chondroitin sulfate, pentosan polysulfate and aminopropionitrile); corticosteroids (such as 11-dehydrocorticosterone, 11-deoxycorticosterone, 11-deoxycortisol, 11-ketoprogesterone, 11β-hydroxypregnenolone, 11β-hydroxyprogesterone, 11β,17α,21-trihydroxypregnenolone, 17α,21-dihydroxypregnenolone, 17α-hydroxypregnenolone, 17α-hydroxyprogesterone, 18-hydroxy-11-deoxycorticosterone, 18-hydroxycorticosterone, 18-hydroxyprogesterone, 21-deoxycortisol, 21-deoxycortisone, 21-hydroxypregnenolone (prebediolone), aldosterone, corticosterone (17-deoxycortisol), cortisol (hydrocortisone), cortisone, pregnenolone, progesterone, flugestone (flurogestone), fluorometholone, medrysone (hydroxymethylprogesterone), prebediolone acetate (21-acetoxypregnenolone), chloroprednisone, cloprednol, difluprednate, fludrocortisone, fluocinolone, fluperolone, fluprednisolone, loteprednol, methylprednisolone, prednicarbate, prednisolone, prednisone, tixocortol, triamcinolone, alclometasone, beclometasone, betamethasone, clobetasol, clobetasone, clocortolone, desoximetasone, dexamethasone, diflorasone, difluocortolone, fluocortolone, flumethasone, fluocortin, fluocortolone, fluprednidene, fluticasone, fluticasone furoate, halometasone, meprednisone, mometasone, mometasone furoate, paramethasone, prednylidene, rimexolone, ulobetasol (halobetasol), amcinonide, budesonide, ciclesonide, deflazacort, desonide, formocortal fluclorolone acetonide (flucloronide), fludroxycortide (flurandrenolone, flurandrenolide), flunisolide, fluocinolone acetonide, fluocinonide, halcinonide and triamcinolone acetonide); quinolines (such as oxycinchophen); gold preparations (such as sodium aurothiomalate, sodium aurothiosulfate, auranofin, aurothioglucose and aurotioprol); penicilamine and similar agents (such as bucillamine); and antihistamines (such as akrivastin, alimemazin, antazolin, astemizol, azatadin, azelastin, bamipin, bilastin, bromodiphenhydramine, bromfeniramin, buklizin, cetirizin, cinnarizine, cyklizin, cyproheptadine, deptropine, desloratadin, dexbromfeniramin, dexklorfeniramin, difenylpyralin, dimenhydrinat, dimetinden, doxylamin, ebastin, epinastin, fenindamin, feniramin, fexofenadin, histapyrrodin, hydroxietylprometazin, isotipendyl, karbinoxamin, ketotifen, kifenadin, klemastin, klorcyklizin, klorfenamin, klorfenoxamin, kloropyramin, levocetirizin, loratadin, mebhydrolin, mekitazin, meklozin, mepyramin, metapyrilen, metdilazin, mizolastin, oxatomide, oxomemazine, pimetixen, prometazin, pyrrobutamin, rupatadin, sekifenadin, talastin, tenalidin, terfenadin, tiazinam, tietylperazin, tonzylamin, trimetobenzamid, tripelennamin, triprolidine and tritokvalin). Combinations of any one or more of the above mentioned antiinflammatory agents may be used.

Preferred antiinflammatory agents include non-steroidal anti-inflammatory drugs, such as diclofenac, ketoprofen, meloxicam, aceclofenac, flurbiprofen, parecoxib, ketoralac tromethamine or indomethacin.

Subjects may receive (or may already be receiving) one or more of the aforementioned co-therapeutic and/or antiinflammatory agents, separate to a formulation of the invention, by which we mean receiving a prescribed dose of one or more of those other therapeutic agents, prior to, in addition to, and/or following, treatment with a formulation of the invention.

When azacitidine/salts thereof are ‘combined’ with such other therapeutic agents, the active ingredients may be administered together in the same formulation, or administered separately (simultaneously or sequentially) in different formulations (hereinafter referred to as ‘combination products’).

Such combination products provide for the administration of azacitidine in conjunction with the other therapeutic agent, and may thus be presented either as separate formulations, wherein at least one of those formulations is a formulation of the invention, and at least one comprises the other therapeutic agent in a separate formulation, or may be presented (i.e. formulated) as a combined preparation (i.e. presented as a single formulation including azacitidine/salt and the other therapeutic agent).

In this respect another therapeutic agent may be co-presented with azacitidine at an appropriate dose in one or more of the cores that form part of a formulation of the invention as hereinbefore described, or may be formulated using the same or a similar process for coating to that described hereinbefore for azacitidine, which may allow for the release of the other therapeutic agent over the same, or over a different timescale. Thus, there is further provided a pharmaceutical formulation of the invention that further comprises a therapeutic agent that is useful in the treatment of a cancers, such as MDS as hereinbefore defined, and/or an antiinflammatory agent;

In such formulations of the invention, the further therapeutic agent may be included by:
(1) formulating along with the azacitidine within the solid cores of a formulation of the invention (which formulation is hereinafter referred to as a ‘combined core preparation’); or
(2) dissolving it, and/or suspending it, within the aqueous carrier system of a formulation of the invention (which formulation is hereinafter referred to as a ‘combination preparation’).

In embodiment (2) above, the other therapeutic agent may be presented in a formulation of the invention in any form in which it is separate to the azacitidine-containing cores. This may be achieved by, for example, dissolving or suspending that active ingredient directly in the aqueous medium of a formulation of the invention, or by presenting it in a form in which its release can, like the azacitidine, also be controlled following injection.

The latter option may be achieved by, for example, providing the other therapeutic agent in the form of additional particles suspended in the aqueous carrier system of formulation of the invention, which additional particles have a weight-, number-, or volume-based mean diameter that is between amount 10 nm and about 700 μm, and comprise cores comprising the therapeutic agent that is useful in the treatment of a cancers, such as MDS as hereinbefore defined, and/or the antiinflammatory agent, which cores are coated, at least in part, by one or more coating materials as hereinbefore described (which formulation is hereinafter referred to as a ‘combination suspension’).

There is further provided a pharmaceutical formulation of the invention that is in the form of a kit of parts comprising components:
(A) a pharmaceutical formulation of the invention; and
(B) a pharmaceutical formulation, comprising a therapeutic agent that is useful in the treatment of a cancer, such as MDS as hereinbefore defined, and/or an antiinflammatory agent,
which Components (A) and (B) are each provided in a form that is suitable for administration in conjunction with the other.

Although Component (B) of a kit of parts as presented above may be different in terms its chemical composition and/or physical form from Component (A) (i.e. a formulation of the invention), it may also be in a form that is essentially the same or at least similar to an azacitidine-containing formulation of the invention, that is in the form of a plurality of particles suspended in an (e.g. aqueous) carrier system, which particles:
(a) have a weight-, number-, or volume-based mean diameter that is between amount 10 nm and about 700 μm; and
(b) comprise solid cores comprising that other therapeutic agent, which cores are coated, at least in part, by one or more coatings of (e.g. inorganic) material.

In addition, although, in such preferred kits of parts, and the combination suspensions presented under embodiment (2) above, the coated cores comprising the other therapeutic agent may be different in terms of their chemical composition(s) and/or physical form(s), it is preferred that the coating of inorganic material that is employed is the same or similar to that employed in azacitidine-containing formulations of the invention, which means that the other therapeutic agent is coated by one or more inorganic coatings as hereinbefore described, for example one or more inorganic coating materials comprising one or more metal-containing, or metalloid-containing, compounds, such as a metal, or metalloid, oxide, for example iron oxide, titanium dioxide, zinc sulphide, more preferably zinc oxide, silicon dioxide and/or aluminium oxide, which coating materials may (on an individual or a collective basis) consist essentially (e.g. are greater than about 80%, such as greater than about, 90%, e.g. about 95%, such as about 98%) of such oxides, and more particularly inorganic coatings comprising a mixture of:
(i) zinc oxide; and
(ii) one or more other metal and/or metalloid oxides,
wherein the atomic ratio ((i):(ii)) is at least about 1:6 and up to and including about 6:1.

Preferably, the atomic ratio ((i):(ii)) is at least about 1:1 and up to and including about 6:1.

In any event, and for the avoidance of doubt, all aspects, including preferred aspects, disclosed and/or claimed herein for in azacitidine-containing formulations of the invention are equally applicable as aspects and/or preferences for coated cores comprising one or more of the further therapeutic agents described above. For the avoidance of doubt, such aspects, preferences and features, alone or in combination, are hereby incorporated by reference to these aspects of the invention.

All combination products, including combined core preparations, combination suspensions and kits of parts described above may thus be used in human medicine and, in particular, any indication in which azacitidine is either approved for use in, or otherwise known to be useful in, such as cancers and MDS as hereinbefore defined.

In certain instances, such additional therapeutic agents, including some of those that are useful in the treatment of e.g. MDS, may be termed ‘standard of care’ in relation to a particular condition. The term ‘standard of care’ will be understood by the skilled person to include a treatment processes that a clinician should, and/or is expected to, follow for certain types of patients, illnesses and/or clinical circumstances. In certain new or poorly-understood conditions, standard of care may change and/or develop over time.

According to a further aspect of the invention, there is provided a method of making a kit of parts as defined above, which method comprises bringing Component (A), as defined above, into association with a Component (B), as defined above, thus rendering the two components suitable for administration in conjunction with each other.

By bringing the two components ‘into association with’ each other, we include that Components (A) and (B) of the kit of parts may be:
(i) provided as separate formulations (i.e. independently of one another), which are subsequently brought together for use in conjunction with each other in combination treatment; or
(ii) packaged and presented together as separate components of a ‘combination pack’ for use in conjunction with each other in combination treatment.

Thus, there is further provided a kit of parts as hereinbefore defined in which Components (A) and (B) are packaged and presented together as separate components of a combination pack, for use in conjunction with each other in combination treatment, as well as a kit of parts comprising:
(I) one of Components (A) and (B) as defined herein; together with
(II) instructions to use that component in conjunction with the other of the two components.

As alluded to above, the kits of parts described herein may comprise more than one formulation including an appropriate quantity/dose of azacitidine/salt, and/or more than one formulation including an appropriate quantity/dose of the other therapeutic agent, in order to provide for repeat dosing as hereinbefore described.

In this respect, with respect to the kits of parts as described herein, by ‘administration in conjunction with’, we include that Components (A) and (B) of the kit are administered, sequentially, separately and/or simultaneously, over the course of treatment of the condition.

Thus, the term ‘in conjunction with’ includes that one or other of the two formulations may be administered (optionally repeatedly) prior to, after, and/or at the same time as, administration of the other component. When used in this context, the terms ‘administered simultaneously’ and ‘administered at the same time as’ include that individual doses of azacitidine/salt and other therapeutic agent are administered within 48 hours (e.g. 24 hours) of each other.

In respect of any of the above combination products according to the invention, the respective formulations are administered (or, in the case of the kit of parts, the two components are administered, optionally repeatedly, in conjunction with each other) in a manner that may enable a beneficial effect for the subject, that is greater, over the course of the treatment of the condition, than if a formulation (e.g. a formulation of the invention) comprising azacitidine/salt alone is administered (e.g. repeatedly, as described herein) in the absence of the other component, over the same course of treatment.

Determination of whether a combination product provides a greater beneficial effect in respect of, and over the course of treatment will depend upon the condition to be treated and/or its severity, but may be achieved routinely by the skilled person.

For example, a physician may initially administer a formulation of the invention comprising alone to treat a patient with MDS, and then find that that person exhibits an inflammatory response (which may be caused by the active ingredient per se and/or by any other component of the formulation).

The physician may then administer one or more of:
Component (B) of a kit of parts as described above,
a combined core preparation,
a combination preparation, and/or
a combination suspension
as described above, any of which comprises an antiinflammatory agent as hereinbefore described.

The other active ingredients/therapeutic agents mentioned above that may be employed in combination products according to the invention may be provided in the form of a (e.g. pharmaceutically-acceptable) salt, including any such salts that are known in the art and described for the drugs in question to in the medical literature, such as Martindale—The Complete Drug Reference, 38^{th} Edition, Pharmaceutical Press, London (2014) and the documents referred to therein (the relevant disclosures in all of which documents are hereby incorporated by reference).

The amount of the other active ingredient/therapeutic agent that may be employed in combination products according to the invention must be sufficient so exert its pharmacological effect.

Doses of such other active ingredients that may be administered to a patient should thus be sufficient to affect a therapeutic response over a reasonable and/or relevant timeframe. One skilled in the art will recognize that the selection of the exact dose and composition and the most appropriate delivery regimen will also be influenced by not only the nature of the other active ingredient, but also inter alia the pharmacological properties of the formulation, the route of administration, the nature and severity of the condition being treated, and the physical condition and mental acuity of the recipient, as well as the age, condition, body weight, sex and response of the patient to be treated, and the stage/severity of the disease, as well as genetic differences between patients.

As administration of formulations of the invention may be continuous or intermittent (e.g. by bolus injection), dosages of such other active ingredients may also be determined by the timing and frequency of administration.

In any event, the medical practitioner, or other skilled person, will be able to determine routinely the actual dosage of any particular additional active ingredient, which will be most suitable for an individual patient, and doses of the relevant additional active ingredients mentioned above include those that are known in the art and described for the drugs in question to in the medical literature, such as Martindale—The Complete Drug Reference, 38^{th} Edition, Pharmaceutical Press, London (2014) and the documents referred to therein, the relevant disclosures in all of which documents are hereby incorporated by reference.

The use of formulations of the invention may control the dissolution rate of azacitidine and affect the pharmacokinetic profile by reducing any burst effect as hereinbefore defined (e.g. a concentration maximum shortly after administration), and/or by reducing Cₘₐₓ in a plasma concentration-time profile.

Formulations of the invention may also provide a release and/or pharmacokinetic profile that increases the length of release of azacitidine from the formulation.

These factors not only reduce the frequency at or over which the formulation needs to be administered to a MDS sufferer, but also allows the sufferer more time as an out-patient, and so to have a better quality of life.

The formulation of the invention also has the advantage that by controlling the release of active ingredient at a steady rate over a prolonged period of time, a lower daily exposure to e.g., a cytotoxic drug is provided, which is expected to reduce unwanted side effects.

The formulations and processes described herein may also have the advantage that, in the treatment of the relevant conditions, they may be more convenient for the physician and/or patient than, be more efficacious than, be less toxic than, have a broader range of activity than, be more potent than, produce fewer side effects than, or that it may have other useful pharmacological properties over, any similar treatments known in the prior art.

Wherever the word ‘about’ is employed herein, for example in the context of amounts (e.g. numbers, concentrations, dimensions (sizes and/or weights), doses, time periods, pharmacokinetic parameters, etc.), relative amounts (percentages, weight ratios, size ratios, atomic ratios, aspect ratios, proportions, factors, fractions, etc.), relative humidities, lux, temperatures or pressures, it will be appreciated that such variables are approximate and as such may vary by ±15%, such as ±10%, for example ±5% and preferably ±2% (e.g. ±1%) from the numbers specified herein. This is the case even if such numbers are presented as percentages in the first place (for example ‘about 15%’ may mean±15% about the number 10, which is anything between 3.5% and 11.5%).

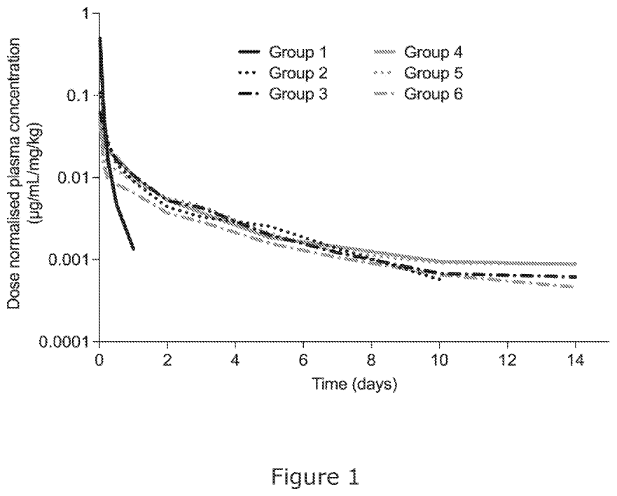

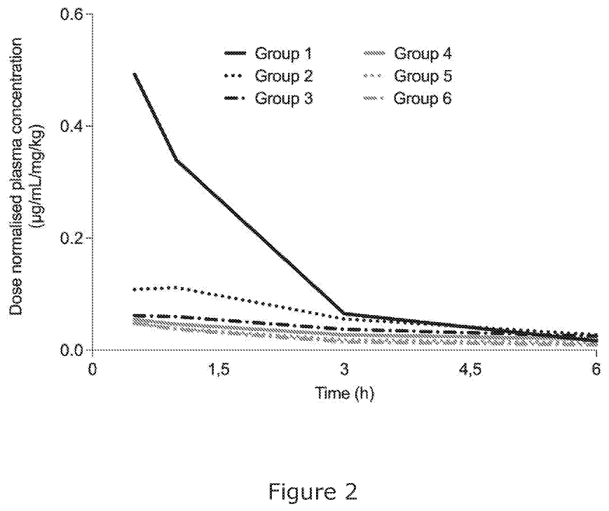

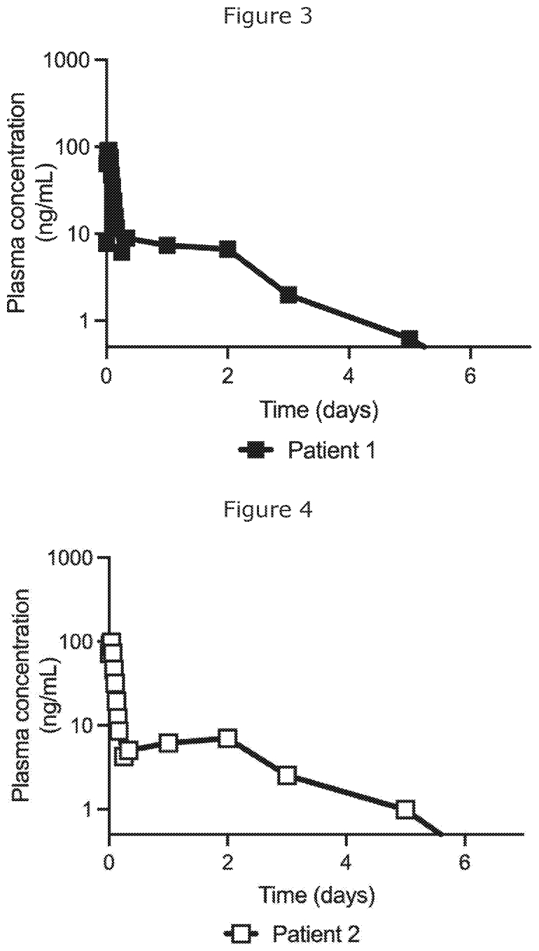

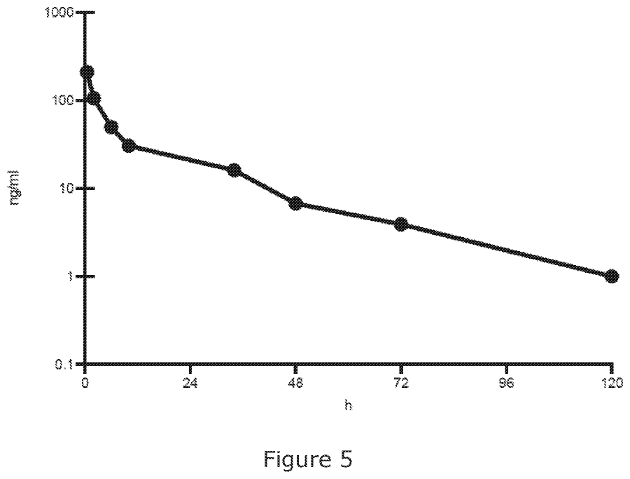

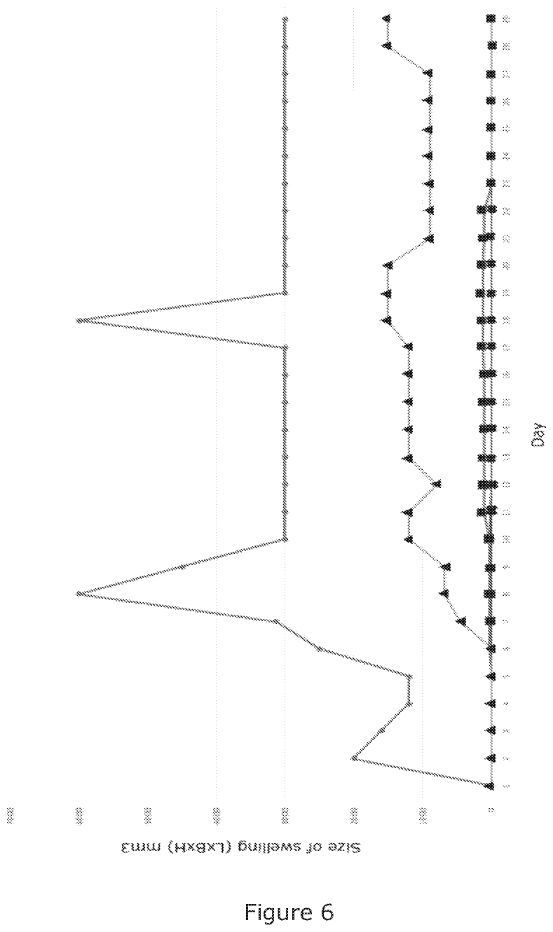

The invention is illustrated, but in no way limited, by the following examples with reference to the attached figures in which FIGS. 1 and 2 show dose-normalised plasma concentrations of azacitidine over different time periods, following subcutaneous administration to rats of various prepared as described hereinafter; FIGS. 3 and 4 show plasma concentration-time profiles for two patients administered azacitidine according to a treatment protocol in a clinical trial as described in Example 10 below; FIG. 5 shows a plasma concentration-time profiles of azacitidine for a minipig following the subcutaneous administration of a formulation of the invention; and FIG. 6 shows the positive impact on local inflammatory response in terms of size of swelling of subcutaneously co-administering formulations of the invention along with mixed oxide coated microparticles comprising the antiinflammatory agent, indomethacin.

### EXAMPLES

### Example 1

### Mixed Oxide Coated Azacitidine Microparticles I

Samples of microparticles of azacitidine (MSN Labs, India) were prepared by jet-milling. The particle size distribution, as determined by laser diffraction, was as follows: D₁₀ 2.9 μm; D₅₀ 7.9 μm; D₉₀ 23.2 μm.

The powder was loaded to an ALD reactor (Picosun, SUNALE™ R-series, Espoo, Finland) where 24 ALD cycles were performed at a reactor temperature of 50° C. The coating sequence was three ALD cycles employing diethyl zinc and water as precursors for three ALD cycles, followed by one cycle of trimethylaluminium and water, repeated six times, to forming a mixed oxide layer of with a atomic ratio of zinc:aluminium of 3:1. The first layer was between about 4 and about 8 nm in thickness (as estimated from the number of ALD cycles).

The powder was removed from the reactor and deagglomerated by means of forcing the powder through a polymeric sieve with a 20 μm mesh size using a sonic sifter.

The resultant deagglomerated powder was re-loaded into the ALD reactor and further 24 ALD cycles were performed as before, forming a second layer of mixed oxide at the aforementioned ratio, followed by extraction from the reactor and deagglomeration by means of sonic sifting as above, followed by reloading to form a third layer, deagglomeration and then reloading to form a final, fourth layer.

To determine the drug load (i.e. w/w % of azacitidine in the powder), HPLC (Prominence-i (Shimadzu, Japan) equipped with a diode array detector (Shimadzu, Japan) set at 223 nm was employed using a 4.6×250 mm, 3 μm particles, CIS column (Luna, Phenomenex, USA)). The nanoshell coatings were dissolved in 5 M phosphoric acid in DMSO and the slurry was diluted with DMSO, before filtration (0.2 μm RC, Lab Logistics Group, Germany) and further analyzed with HPLC (n=2). The drug load was determined as 88.9%.

### Example 2

### Mixed Oxide Coated Azacitidine Microparticles II

The same procedure as described in Example 1 was conducted to produce microparticles coated with mixed oxide coating comprising a atomic ratio of zinc:aluminium of 2:1.

The coating sequence was two ALD cycles employing diethyl zinc and water as precursors, followed by one cycle of trimethylaluminium and water, repeated ten times, removal of the coated powder from the reactor, deagglomeration, reloading and repeating the same coating sequence, removal, deagglomeration until 4 sets of 30 cycles in total had been provided.

The drug load was determined as 86.6%.

### Example 3

### Mixed Oxide Coated Azacitidine Microparticles III

The same procedure as described in Example 1 was conducted, but this time a thicker mixed oxide coating comprising a atomic ratio of zinc:aluminium of 3:1 was produced by conducting 6 sets of 24 ALD cycles.

The drug load was determined as 80.7%.

### Comparative Example 4

### Aluminium Oxide Coated Azacitidine Microparticles

The same microparticles that were coated with the mixed oxide coating as described in Example 1 were coated with a pure aluminium oxide coating, 7 ALD cycles were performed prior to removing the coated powder from the reactor and deagglomerated as described in Example 1. The resultant deagglomerated powder was reloaded into the ALD reactor and subjected to a further 7 ALD cycles, followed by extraction, deagglomeration and reloading for 7 cycles was repeated twice thereafter, followed by reloading and subjecting to 2 times 14 cycles.

The drug load was determined as 91.3%.

### Comparative Example 5

### Suspension of Azacitidine Microparticles in Vehicle

The same microparticles that were coated with the mixed oxide coating as described in Example 1 were suspended in a commercially-available aqueous vehicle, Hyonate® vet, which is a veterinary medicinal product used in injecting animals, such as horses, comprising a sterile, isotonic, phosphate buffered solution of 10 mg/mL of sodium hyaluronate (pH 7.4).

The concentration of azacitidine in the formulation was 10 mg/mL, which accords to 5 mg/kg body weight of a Sprague-Dawley rat.

### Comparative Example 6

### Suspension of Aluminium Oxide Coated Azacitidine Microparticles in Vehicle

The coated microparticles from Comparative Example 4 were suspended in a commercially-available aqueous vehicle, Hyonate® vet to a concentration of azacitidine in the formulation of 27 mg/mL, which accords to 13.5 mg/kg body weight of a Sprague-Dawley rat.

### Example 7

### Formulations of the Invention I

Three suspensions of coated microparticles of azacitidine (prepared according to the processes described in Examples 1, 2 and 3 above) was suspended in Hyonate vet.

A further suspension of coated microparticles of azacitidine (prepared according to the process described in Example 3 above) was suspended in an aqueous vehicle comprising 0.1% (w/w) of Polysorbate 20 and 0.25% (w/w) sodium carboxylmethyl cellulose in a phosphate buffered saline solution (pH 7.4).

In each case, the concentration of azacitidine in the formulation was 27 mg/mL, which accords to 13.5 mg/kg body weight of a Sprague-Dawley rat.

### Example 8

### In Vivo Rat Study

Thirty-eight male Sprague Dawley rats weighing between 266 and 302 g at the day of administration were supplied by Charles River (UK). The animals were divided randomly into six animals per group.

The intended administration area was clipped free from hair prior to injection and the injection site was marked. The suspensions described in Comparative Example 5 (Group 1), Comparative Example 6 (Group 2) and Example 7 (Groups 3 to 6 as identified in Table 1 below) were drawn into a 1 mL BD syringe and single, subcutaneous injections (ca. 0.15 mL) were administered through a 20 G needle (BD microlance) into the flank of each rat. Administration was performed no more than 10 minutes after preparation of the formulations.

TABLE 1

| Group | Description | Dose |
|---|---|---|
| 1 | Uncoated particles in Hyonate (Ex. 5) | 5.0 ± 0.08 |
| 2 | Al₂O₃ coated particles in Hyonate (Ex. 4 and 6) | 13.5 ± 0.23 |
| 3 | Mixed (2:1) coated particles in Hyonate (Ex. 2 | 13.6 ± 0.40 |
| | and 7) | |
| 4 | Mixed (3:1) coated particles in Hyonate (Ex. 1 | 13.5 ± 0.33 |
| | and 7) | |
| 5 | Mixed (3:1) thicker coated particles in Hyonate | 13.7 ± 0.17 |
| | (Ex. 3 and 7) | |
| 6 | Mixed (3:1) thicker coated particles in PBS (Ex. | 13.7 ± 0.30 |
| | 3 and 7) | |

Blood samples (ca 0.2 mL) were collected from the tail vein into K₂EDTA (dipotassium ethylenediaminetetraacetic acid) tubes containing 5 μL of THU (25 μL/mL blood; tetrahydrouridine, which is a competitive cytidine deaminase inhibitor) stabilising agent (1 mg/mL aqueous solution) at the following time-points: 0.5, 1, 3, 6, 12, 24, 48, 72, 120, 168, 240, and 336 h post-dose. Actual sampling times were recorded. As soon as practically possible following blood sampling, plasma was separated by centrifugation (1500 g for 10 min at 4° C.), which was stored at −80° C. until analysis was conducted.

Following study completion, all plasma samples were shipped for analysis having been deep frozen on dry ice. Animals were sacrificed on the last day of the study.

Plasma concentration of azacitidine was determined with LC-MS/MS. Study samples were prepared by pipetting 25 μL of rat plasma into a 96 well plate, adding 25 μL 5% DMF in acetonitrile and 75 μL of an internal standard working solution using the TECAN Genesis liquid handling robot. The 96 well plates were then shaken for 15 minutes and centrifuged. All samples were then injected on a UPLC-MS/MS system. Separation was obtained with an ACQUITY BEH Amide Column, 2.5 μm, 2.1×100 mm, Waters at 25° C. using 10 mM ammonium formiate pH 3.4 in water with 0.125 μM Li Acetate as mobile phase A (MP A) and acetonitrile as mobile phase B (MP B).

Pharmacokinetic analysis of azacitidine in plasma was performed according to standard non-compartmental approach using Microsoft Excel for Mac (16.43, Microsoft, Redmond, Washington, USA). Maximum concentration, Cₘₐₓ, and related time, tₘₐₓ, were the coordinates of the highest concentration of the time course. tₗₐₛₜ was the time of the last detectable concentrations. The area under concentration vs. time curve up to the last detectable concentration, AUCₗₐₛₜ, was calculated using the linear trapezoidal rule.

The dose site on each animal was marked post-dosing and kept free from hair for the duration of the study. Dose site observations were performed at 24, 120, 168, and 336 hours post dosing.

### Results

Dose-normalised plasma concentrations of azacitidine over two weeks after single subcutaneous administration of the various formulations are presented in FIG. 1, with FIG. 2 showing the same plasma concentration profiles over the first six hours. The plasma pharmacokinetic parameters are also presented as mean values for the group of 6 rats (with standard deviations provided in parentheses) in Table 2 below, in which:
dose is expressed in mg/kg body weight of the rat
‘tₘₐₓ’ is the time to peak concentration expressed in hours
‘Cₘₐₓ’ is the maximum concentration found in analysis expressed in μg/mL
‘tₗₐₛₜ’ is the time of the last detectable concentration expressed in hours
‘t_{1/2.z}’ is the terminal half-life expressed in hours
‘AUC_{∞}’ is the area under concentration vs. time curve up to infinite time expressed in μg*h/mL
‘F’ is the relative bioavailability expressed as a percentage
‘Cₘₐₓ/D’ is the maximum concentration normalized to 1 mg/kg expressed in μg/mL/mg/kg body weight of the rat
‘AUCₗₐₛₜ/D’ is the area under blood concentration vs. time curve up to the last detectable concentration normalized to 1 mg/kg expressed in μg*h/mL/mg/kg body weight of the rat
‘AUC_{∞}/D’ is the area under concentration vs. time curve LIP to infinite time normalized to 1 mg/kg expressed in μg*h/mL/mg/kg body weight of the rat
‘Fr. Rel_{.0-12n}’ is the fraction released during the first twelve hours of the area under concentration vs. time curve up to infinite time expressed as a percentage.

TABLE 2

| Parameter | Group 1 | Group 2 | Group 3 | Group 4 | Group 5 | Group 6 |
|---|---|---|---|---|---|---|
| Dose | 5.0 | 13.5 | 13.6 | 13.5 | 13.7 | 13.7 |
| | (0.080) | (0.23) | (0.40) | (0.33) | (0.17) | (0.30) |
| tₘₐₓ | 0.50 | 0.83 | 0.75 | 0.50 | 0.50 | 0.50 |
| | (0.0) | (0.26) | (0.27) | (0.0) | (0.0) | (0.0) |
| Cₘₐₓ | 2.5 | 1.5 | 0.87 | 0.74 | 0.67 | 0.66 |
| | (0.11) | (0.096) | (0.071) | (0.055) | (0.096) | (0.11) |
| tₗₐₛₜ | 24 | 240 | 336 | 336 | 336 | 320 |
| | (0.0) | (0.0) | (0.0) | (0.0) | (0.0) | (39) |
| t_{1/2, z} | 1.6 | 58 | 65 | 140 | 102 | 77 |
| | (0.40) | (10) | (21) | (88) | (64) | (24) |
| AUC_{∞} | 4.9 | 17 | 17 | 18 | 16 | 11 |
| | (0.32) | (1.2) | (1.6) | (2.9) | (2.1) | (1.6) |
| F | 100 | 129 | 128 | 138 | 120 | 81 |
| | (5.0) | (12) | (9.0) | (21) | (15) | (11) |
| Cₘₐₓ/D | 0.49 | 0.11 | 0.064 | 0.055 | 0.049 | 0.048 |
| | (0.016) | (0.006) | (0.005) | (0.005) | (0.007) | (0.008) |
| AUCₗₐₛₜ/D | 0.98 | 1.2 | 1.2 | 1.2 | 1.0 | 0.75 |
| | (0.049) | (0.12) | (0.068) | (0.10) | (0.12) | (0.10) |
| AUC_{∞}/D | 0.98 | 1.3 | 1.3 | 1.4 | 1.2 | 0.80 |
| | (0.049) | (0.11) | (0.089) | (0.21) | (0.15) | (0.10) |
| Fr. Rel._{0-12 h} | 96 | 39 | 29 | 22 | 19 | 22 |
| | (0.40) | (1.8) | (1.5) | (2.4) | (1.5) | (1.8) |

It can be seen that the plasma concentration profile was comparable for Groups 3 to 6, with maximal plasma concentration (Cₘₐₓ) reached within the first hour followed by a slow but steady decline in concentrations over the 14 day study period.

Group 2 had higher initial drug release compared with the mixed oxide coating formulations.

Comparing Group 5 to Group 6, it can be seen that the PBS-based vehicle resulted in a slightly lower AUC compared to when Hyonate vet was used, although the respective pharmacokinetic profiles were largely comparable.

Groups 2 to 6 also demonstrated relative bioavailabilities (F) that were comparable with the uncoated azacitidine, in stark contrast to our previous studies on aqueous suspension of zinc oxide coated azacitidine, which show lower F values.

Cₘₐₓ was lower for Groups 2 to 6 compared to uncoated azacitidine (Group 1), with approximately one fifth of the dose being released during the first day. When normalised for dose, the difference is about an order of magnitude. Also, the residual area (correlating to unreleased drug) following the last sampling time (336 hours post-dosing) was <12%.

The results for Group 2, showed a slightly different profile characterized by a larger fraction of the dose released during the first day, a higher Cₘₐₓ, and a shorter duration.

In summary, Groups 3 to 6 demonstrated a prolonged-release profile, which differs from the rapid decline following administration of uncoated azacitidine. Similarly, advantageous plasma concentration-time profiles were observed for all of the formulations of the invention.

### Example 9

### Mixed Oxide Coated Azacitidine Microparticles IV

The same procedure as described in Example 1 was conducted, with the exception that microparticles of azacitidine had particle size distribution as follows: D₁₀ 1.2 μm; D₅₀ 3.8 μm; D₉₀ 11.3 μm. The drug load was determined as 81.3%.

### Example 10

### Phase Ia Clinical Trial

An open pilot Phase Ia clinical study to assess the pharmacokinetics, tolerability, and safety of the coated azacitidine microparticles from Example 9 above suspended in Hyonate vet (Boehringer Ingelheim Animal Health; an aqueous solution comprising sodium hyaluronate (10 mg/mL), sodium chloride (8.5 mg/mL), disodium phosphate (0.223 mg/mL), sodium dihydrogen monohydrate phosphate (40 μg/mL), HCl and NaOH for pH adjustment), and administered as a subcutaneous injection for the treatment of intermediate 2 or higher-risk MDS, CMML, or AML, in patients already on treatment with azacitidine, was carried out.

Pharmacokinetic parameters including AUC₀₋₂₄ₕ, AUC₀₋ₗₐₛₜ, AUC_{0-∞}, Cₘₐₓ, Cₗₐₛₜ, terminal t_{1/2}, volume of distribution V_{d} and clearance were measured.

Local tolerance were measured by inspection of injection sites. Pain, tenderness erythema/redness, and induration/swelling were assessed by a four-grade scale, in which 1 is considered mild and 4 is considered potentially life threatening.

It was intended to include 6 patients in the study, which would consist of a screening phase, a treatment phase, interim analysis, and a follow-up phase.

Inclusion criteria included:
a Written informed consent prior to any study specific procedures.
patients≥18 years of age
Body Mass Index (BMI)≥19 and ≤32 kg/nm² BSA at screening
current treatment with azacitidine corresponding to 100 mg/m² BSA×5 or ×4 per treatment cycle for at least six cycles for diagnosed:
   a. intermediate-2 and high-risk myelodysplastic syndromes (MDS) according to the International Prognostic Scoring System (IPSS)
   b. chronic myelomonocytic leukemia (CMML) with 10-29% marrow blasts
   c. acute myeloid leukemia (AML) according to World Health Organization (WHO) classification
Eastern Cooperative Oncology Group (ECOG) performance status of 0, 1, or 2
Recovery of Hematology and Clin. Chemistry assessment according to clinical practices at the start of the last azacitidine treatment cycle before the screening visit
Female subjects of non-childbearing potential (defined as pre-menopausal females with a documented tubal ligation or hysterectomy or bilateral oophorectomy; or as post-menopausal females defined as 12 months' amenorrhea)
Male patients agreed to use an adequate method of contraception
Willingness and ability to comply with study procedures, visit schedules, study restrictions, and requirements
   Exclusion criteria included:
a That the patient has participated in any other investigational/interventional trial including an investigational drug within 30 days (or five half-lives of the study drug prior to screening, whichever is longer) prior to screening
Diagnosis of malignant disease within the previous 5 years (excluding basal cell carcinoma of the skin without complications, in-situ carcinoma of the cervix or breast, or other local malignancy excised or irradiated with a high probability of cure)
Any significant medical condition, laboratory abnormality, or psychiatric illness that would prevent the patient from participating in the study
History of alcohol abuse or drug abuse within the past 12 months
Any condition including the presence of laboratory abnormalities, which places the patient at unacceptable risk if he/she were to participate in the study
Other reasons for non-suitability for participation, as judged by the Investigator.

The duration for a patient in the study was intended to be approximately 2-3 months. This timeframe consisted of a 3-4 week screening period, followed by approximately four weeks in the treatment phase, which comprised, from Day 1 to Day 4, of daily injections of uncoated azacitidine (Vidaza® or generic azacitidine (Mylan), freeze dried powder for injection suspended in water for injection), both 100 mg/m² BSA, 25 mg/mL).

Samples were taken for pharmacokinetic analysis on Day 4 (before commencement of study drug). The mean maximum plasma concentration (Cₘₐₓ) was 562 ng/mL and occurred after a tₘₐₓ of 0.433 hours. The mean half-life was 6.82 hours. The mean AUC_{inf} was 1120 ng h/mL.

On Day 5, a single administration of the study drug suspension as described above was given (100 mg/m² BSA, 100 mg/mL). Samples were to be taken for pharmacokinetic analysis on each of Days 5 to 8, and then Days 10, 12, 15, 17 and 19.

It was also intended that, after the treatment phase, the last azacitidine dose would be replaced by a single dose of an azacitidine comparator (as above), and a follow-up visit scheduled to take place on the same day.

However, after two enrolled patients were subjected to the treatment phase, the study was put on hold after a meeting of an internal safety committee, which was charged with the review of safety data.

It was noted that induration and inflammation (redness and light pain), classified as moderate at the injection site was exhibited in both patients. It was decided not to enroll any more patients in the study and to request additional expert review/analysis(es) of biopsy results from the two patients.

The plasma concentration time curves for the two patients after administration of study drug are nevertheless presented in FIGS. 3 and 4, respectively, showing a clear steady-state sustained release of azacitidine from the injected study drug formulations. The plasma concentration time curve is presented in the graphs on a semi-logarithmic scale (squares). The mean maximum plasma concentration (Cₘₐₓ) was 94.8 ng/mL and occurred after (Tₘₐₓ) 1.02 hours. The mean half-life was 15.2 hours. The mean AUC_{inf} was 495 ng h/mL.

### Example 11

### Combination Formulations of the Invention

Various formulations of the invention comprising combinations of azacitidine and indomethacin are prepared as follows:

(A) A blended mixture of microparticles of azacitidine and indomethacin in a weight-ratio between 100:1 to 1:10, are prepared by jet-milling. The particle size distribution, as determined by laser diffraction, is an average particle size of between 0.1 and 100 μm.

The resultant powder is coated by ALD as described in Example 1 above and formulated in a vehicle and used for treatment of patients suffering from MDS as described in Example 10 above.

(B) Microparticles comprising an e.g. co-precipitated mixture of azacitidine and indomethacin in a weight-ratio between 100:1 to 1:10, are prepared. The particle size distribution, as determined by laser diffraction, is an average particle size of between 0.1 and 100 μm.

The microparticles are coated by ALD as described in Example 1 above and formulated in a vehicle and used for treatment of patients suffering from MDS as described in Example 2 above.

(C) Two sets of microparticle samples are separately prepared by jet-milling. A first set comprises azacitidine and a second set comprises indomethacin. The particle size distribution in both sets of samples, as determined by laser diffraction, is between 0.1 and 100 μm.

Both sets of samples are coated separately by ALD as described in Example 1 above and are mixed in a formulation wherein the weight-ratio between powders of the first and the second set is between 100:1 to 1:10.

The mixed powder is formulated in a vehicle and used for treatment of patients suffering from MDS as described in Example 10 above.

(D) Samples of azacitidine is prepared as described in Example 1 are formulated in a vehicle as described in Example 10 above. An additional formulation comprising indomethacin particles is prepared in the same way.

Both formulations are used for injected at essentially the same time on different sites as described in Example 10 above for treatment of patients suffering from MDS. The dose ratio with respect to weight of azacitidine and indomethacin in the two different injections is between 100:1 and 1:10.

(E) Coated particles of azacitidine are prepared essentially as described in Example 1 above and are formulated in a vehicle as described in Example 10 above, further comprising dissolved and/or suspended indomethacin.

The formulation is used for treatment of patients suffering from MDS as described in Example 10 above.

In all of cases (A) to (E) above, any inflammatory reactions at the site of the subcutaneous administration (and formed depot) are suppressed by the antiinflammatory properties of indomethacin.

### Example 12

### Mixed Oxide Coated Azacitidine Microparticles V

The same procedure as described in Examples 1 and 9 was conducted to produce coated azacitidine microparticles with a drug load that was determined as 80.1%.

### Example 13

### Mixed Oxide Coated Azacitidine Microparticles VI

Essentially the same procedure as described in Examples 1 and 9 was conducted, except that 30 ALD cycles were performed at a reactor temperature of 50° C., with a coating sequence of two ALD cycles employing diethyl zinc and water as precursors followed by one cycle of trimethylaluminium and water, repeated ten times, to forming a mixed oxide layer of with a atomic ratio of zinc:aluminium of 2:1. The first layer was estimated as between about 5 and about 10 nm in thickness.

The powder was removed from the reactor and deagglomerated by means of forcing the powder through a polymeric sieve with a 20 μm mesh size using a sonic sifter and then the deagglomerated powder re-loaded into the ALD reactor and a further 30 ALD cycles performed as above, forming a second layer of mixed oxide at the same ratio, extraction from the reactor and deagglomeration by using sonic sifting as above, with the process being repeated to form a total of eight layers.

The drug load was determined as 69.1%.

### Comparative Example 7

### Mixed Oxide Coated Indomethacin Microparticles

Samples of microparticles of indomethacin (Recce Pharmaceuticals, Australia) were prepared by jet-milling. The particle size distribution, as determined by laser diffraction, was as follows: D₁₀ 1.2 μm; D₅₀ 3.8 μm; D₉₀ 11.3 μm.

The same ALD coating and intermittent deagglomeration process as described in Example 1 was conducted to form coated indomethacin microparticles with four separate mixed oxide layers with a atomicratio of zinc:aluminium of 3:1.

The drug load was determined as 81%.

### Comparative Example 8

### Mixed Oxide Coated Lactose Microparticles

Samples of microparticles of lactose (InhaLac® 400, Meggle, Germany) was used. The nominal particle size distribution was as follows: D₁₀ 0.8-1.6 μm; D₅₀ 4.0-11.0 μm; D₉₀ 15-35.0 μm.

The powder was loaded to an ALD reactor (Picosun, SUNALE™ R-series, Espoo, Finland) where 48 ALD cycles were performed at a reactor temperature of 50° C. The coating sequence was three ALD cycles employing diethyl zinc and water as precursors for three ALD cycles, followed by one cycle of trimethylaluminium and water, repeated twelve times, to forming a mixed oxide layer of with a atomic ratio of zinc:aluminium of 3:1. The first layer was between about 8 and about 16 nm in thickness (as estimated from the number of ALD cycles).

The powder was removed from the reactor and deagglomerated by means of forcing the powder through a polymeric sieve with a 20 μm mesh size using a sonic sifter.

The resultant deagglomerated powder was re-loaded into the ALD reactor and further 48 ALD cycles were performed as before, forming a second layer of mixed oxide at the aforementioned ratio, followed by extraction from the reactor.

The particle size distribution of the coated lactose microparticles, as determined by laser diffraction, was as follows: D₁₀ 2.1 μm; D₅₀ 7.6 μm; D₉₀ 23.4 μm.

### Example 14

### Formulations of the Invention II

The coated microparticles from Example 12 above were suspended in Hyonate vet in a glass vial to give a final concentrations of 100 mg/mL and 200 mg/mL, respectively (referred to hereinafter as ‘Formulation B’ and ‘Formulation E’, respectively).

Coated microparticles from Example 12 above, along with coated indomethacin microparticles from Comparative Example 7 above, were suspended together in Hyonate vet in a glass vial to give a final concentration of each coated active ingredient as set out in Table 3 below, in which the formulations are also identified in the manner they are referred to hereinafter.

TABLE 3

Coated Microparticles Concentration (mg/mL)

| Azacitidine | Indomethacin | Formulation |
|---|---|---|
| 100 | 0 | B |
| 100 | 25 | F |
| 100 | 5 | G |
| 100 | 100 | D |
| 200 | 0 | E |
| 200 | 25 | H |
| 200 | 5 | I |
| 200 | 100 | J |

### Comparative Example 9

### Formulations Comprising Mixed Oxide Coated Indomethacin and Lactose Microparticles

The procedure described in Example 14 above was followed to produce suspensions of coated microparticles of indomethacin (from Comparative Example 7 above) and coated microparticles of lactose (from Comparative Example 8 above) at final concentrations of 100 mg/mL in 2.2 mL of Hyonate vet.

These suspensions were respectively labelled ‘Formulation C’ (indomethacin) and ‘Formulation A’ (lactose).

### Example 14

### Minipig Study I

The objective of this study (which was carried out at Scantox A/S, Denmark) was to assess the local tolerance and pharmacokinetics of azacitidine formulated according to the invention administered by subcutaneous injection to a minipig, as well as local tolerance following administration of azacitidine formulated according to the invention and indomethacin formulated as described herein.

The minipig was selected as the test model because of its well accepted suitability in this type of study and the close resemblance in skin physiology between humans and minipigs. A staggered dose scheme starting with two doses equivalent to ¼ and ½ of the equivalent human clinical dose before going up to the full dose were chosen to reduce the risk for severe local reactions.

The animal had a body weight of 24.9 kg when allocated to the study and was housed in accordance with EU Directive 2010/63/EU of 22 Sep. 2010 on the protection of animals used for scientific purposes. In short, a standard minipig diet was offered twice daily (morning and afternoon) in an amount of approximately 350 g per meal.

The amount of diet may be adjusted during the course of the study in order to allow a reasonable growth of the animal. A supply of dehydrated grass (Compact Gras, Hartog B. V., Netherlands) was also given daily and the animal had ad libitum access to domestic quality drinking water.

One week prior to start of treatment, the animal was anaesthetised by an intramuscular injection in the neck (1.0 mL/10 kg body weight), and a total of 6 injection sites (approximately 2×2 cm) were tattooed on back of its neck.

The animal was then anaesthetised again 3 days prior to procedure and an ear vein catheter was implanted to take blood samples during the study. For pain treatment during the study, the animal was given an intramuscular injection in the hind leg of meloxicam 5 mg/mL (0.08 mL/kg) just prior to implantation and once daily for the following two days.

The animal received an intravenous injection of 200 mg ampicillin/mL (0.05 mL/kg). The catheter was flushed with 10 mL of sterile saline and locked using 0.5 mL of TauroLock Hep500 (Taurolidin Citrate with 500IE/mL heparin). A stopper, e.g. Bionector IV access system was applied to the luer.

Between blood sampling occasions, TauroLock™ Hep500 will produce a heparin lock in the catheter.

Single doses of study formulations were given by subcutaneous injection in each of the six marked injection sites as set out in Table 4 below.

TABLE 4

| Injection | | Dose Volume | Dose Amount |
|---|---|---|---|
| Site | Formulation | (mL) | (mg) |
| 1 | A | 1.9 | ca. 200* |
| 2 | B | 0.5 | 50 |
| 3 | B | 1.0 | 100 |
| 4 | B | 0.5 | 50 |
| 5 | D | 0.5 | 50 + 50 |
| 6 | C | 0.5 | 50 |

*Estimated

### Local Tolerance

On Day 1 of the study, the animal was anaesthetized and subcutaneously administered Formulations A and B at Injection Sites 1, 2 and 3 in the relevant dose volumes, as set out in Table 4 above. On Day 41 of the study, the animal was anaesthetized and subcutaneously administered Formulations D and C at injection Sites 5 and 6 in the relevant dose volumes, as set out in Table 4 above.

These injections were assessed for local tolerance.

In each case, prior to injection, the relevant sample vials were inverted 3 times just before retracting the sample for each injection to avoid sedimentation of the test material and consequent deviation from the correct dose.

All clinical signs of ill health and any behavioural changes were recorded daily. In addition, close related observations were performed prior to dosing/in relation to dosing and no earlier than 30 minutes after closing and any deviation from normal was recorded.

Injection sites were photographed, scored, and recorded at 30 minutes and 2 and 6 hours post dosing and then daily until no score was presented. From Day 10 and onwards, no photographs were taken, and the injection sites were only scored every second day until no score was present or the study had ended.

Injection Sites 5 and 6 were photographed, scored, and recorded at 30 minutes, and 2 and 6 hours post-dosing and daily until Day 48. Thereafter, no photographs were taken, and the injection sites were only scored twice weekly until no score was present or the study had ended.

Particular attention was paid to haemorrhage, erythema, swelling (with indication/measurement of size) and firmness/induration and necrosis, and any other signs of inflammatory or allergic reactions. Parameters were scored according to the following grading system: 0 (not present), 1 (minimal), 2 (slight), 3 (moderate) and 4 (marked).

On Day 2, blood samples were taken from the animal to assess clinical pathology parameters. An additional sampling took place on Day 5. The animal was fasted overnight before blood samples were taken but water was available.

For haematology, at least 2.5 mL K3 EDTA stabilised blood was taken. From this sample, a back-up smear was prepared and stained with May-Grunwald and Giemsa for possible later manual differential leucocyte count. The smears were not analysed and were discarded upon finalization of the study. For the coagulation tests, 1.8 mL citrate stabilised blood was taken. The parameters, methods and units for the laboratory investigations are presented in Table 5 below.

TABLE 5

| Parameter | Method/Equipment | Units |
|---|---|---|
| Haemoglobin (Hb) | Direct measurement/ABX | mmol/L |
| | Pentra DX120SPS | |
| Red blood cell count (RBC) | Direct measurement/ABX | 10¹²/L |
| | Pentra DX120SPS | |
| Reticulocyte count (RETIC) | Direct measurement/ABX | % and |
| | Pentra DX120SPS | 10¹²/L |
| Haematocrit (HT) | Direct measurement/ABX | L/L |
| | Pentra DX120SPS | |
| Mean cell volume (MCV) | Calculated/ABX Pentra | fL |
| | DX120SPS | |
| Mean cell haemoglobin (MCH) | Calculated/ABX Pentra | fmol |
| | DX120SPS | |
| Mean cell haemoglobin | Calculated/ABX Pentra | mmol/L |
| concentration (MCHC) | DX120SPS | |
| White blood cell count (WBC) | Direct measurement/ABX | 10⁹/L |
| | Pentra DX120SPS | |
| Differential leucocyte count | Direct measurement/ABX | % and |
| (NEUTRO, LYMPHO, EOS, | Pentra DX120SPS(**) | 10⁹/L |
| BASO, MONO) | | |
| Platelet count (Plt) | Direct measurement/ABX | 10⁹/L |
| | Pentra DX120SPS | |
| Activated partial thromboplastin | IL Test ™/ACL ™(*), | sec. |
| time (APTTsyn) | elagic acid as activator | |
| Prothrombin time HS (PT-HS) | IL Test ™/ACL ™(*) | sec. |
| Fibrinogen Clauss (FibC) | IL Test ™/ACL ™(*) | g/L |

(*)Instrumentation Laboratories, Automated Coagulation Laboratory
(**)At the discretion of the technician, individual smears may be manually counted

Approximately 3 mL blood was taken for clinical chemistry in tubes with clotting activator for serum. The parameters, methods and units for the laboratory investigations are presented in Table 6 below.

TABLE 6

| Parameter | Method/Equipment | Units |
|---|---|---|
| Alanine aminotransferase (ALAT) | Cobas 6000 | μkat/L |
| Aspartate aminotransferase (ASAT) | Cobas 6000 | μkat/L |
| Alkaline phosphatase (ALKPH) | Cobas 6000 | μkat/L |
| Bilirubin (total) (TBILI) | Cobas 6000 | μmol/L |
| Gamma-glutamyl transferase (GGT) | Cobas 6000 | μkat/L |
| Cholesterol (CHOL) | Cobas 6000 | mmol/L |
| Triglycerides (TRIG) | Cobas 6000 | mmol/L |
| Carbamide (UREA) | Cobas 6000 | mmol/L |
| Creatinine (CREAT) | Cobas 6000 | μmol/L |
| Glucose (GLUC) | Cobas 6000 | mmol/L |
| Sodium (Na) | Ion selective electrode/ | mmol/L |
| | Cobas 6000 | |
| Potassium (K) | Ion selective electrode/ | mmol/L |
| | Cobas 6000 | |
| Calcium (Ca) | Cobas 6000 | mmol/L |
| Magnesium (Mg) | Cobas 6000 | mmol/L |
| Inorganic phosphorus (P) | Cobas 6000 | mmol/L |
| Chloride (Cl) | Ion selective electrode/ | mmol/L |
| | Cobas 6000 | |
| Protein (total) (PROTEIN) | Cobas 6000 | g/L |
| Albumin (ALB) | Cobas 6000 | g/L |
| Globulin | Calculated | g/L |
| Albumin/Globulin (ALB/G) ratio | Calculated | No unit |

Full-thickness biopsies were taken on Days 3 and 7 from Injection Site 1, and on Days 2 and 6 from injection Sites 2 and 3. A single control biopsy was taken outside of the injection site for comparison at the histopathological evaluation. Full-thickness biopsies were taken on Days 43 and 47 from Injection Sites 5 and 6.

The animal was anaesthetised prior to biopsy collection and, approximately 30 minutes prior to the first sampling of biopsies, an intramuscular injection of methadone 10 mg/mL (0.02 mL/kg) was given to prevent reactions of pain.

Biopsies were collected using a 8 mm punch and were fixed in phosphate buffered neutral 4% formaldehyde. After fixation, the specimens were trimmed and processed. The specimens were embedded in paraffin and cut at a nominal thickness of approximately 5 μm, stained with haematoxylin and eosin and examined under a light microscope. All pathological findings were entered directly into Instem Provantis® (version 9.3.0.0). Histological alterations were graded on a 5-level scale (Minimal, Mild, Moderate, Marked and Severe).

### Pharmacokinetics (PK)

On Day 22 of the study, the animal was anaesthetized and subcutaneously administered Formulation B at Injection Site 4 in the relevant dose volume, as set out in Table 4 above. This injection was assessed for PK parameters.

Prior to injection, the relevant sample vial was inverted 3 times just before retracting the sample as described above for each injection to avoid sedimentation of the test material and consequent deviation from the correct dose.

On the day of closing, blood samples were taken at the following time points: pre-treatment, and 30 min, 2, 6, 10, 24, 48, 72, 120 and 168 hours post-treatment. Blood samples of approximately 3 mL was drawn from the jugular vein/bijugular trunk. The blood was sampled into vacutainers containing K2-EDTA as anticoagulant. The vacutainer was placed in ice water until centrifugation (10 min, 1270 G, +4° C.). Each plasma sample was divided into two aliquots of approx. 0.5 mL and transferred to cryotubes provided by the Sponsor and frozen at −18° C. or below within 90 minutes after collection. The first set of samples were sent on dry ice (approximately −70° C.) for analysis Shipment should be sent without thermologger. The second set of samples were stored at −18° C. or below as back-up samples. The back-up samples were shipped a couple of days after receipt of the primary samples.

Azacitidine in plasma was determined by UPLC-MS/MS. Azacitidine was extracted from plasma by protein precipitation using DMF:Acetonitrile (5:95). After injection on a straight phase chromatographic column, the substance was eluted with an acetonitrile and aqueous gradient and detected with MS/MS.

Individual plasma concentration profiles were subjected to non-compartmental pharmacokinetic analysis using the software PKanalix (version 2020).

When the plasma concentration obtained in the pre-dose sample was below LLOQ, the data point was entered as zero. All other concentrations below LLOQ were entered as half the value of LLOQ (½*LLOQ). Consecutive data points below LLOQ after Tmax was excluded from modelling and analysis.

The maximum plasma concentration (Cmax) and the time when it occurs (Tmax) was estimated by visual inspection of the data.

The area under the curve from time zero to the time point of the last quantifiable concentration (AUC₍₀₋ₜ₎) and the area Linder the curve from time zero to infinity (AUC_{inf}) was calculated according to the linear/log trapezoidal method. If the extrapolated area (AUC(% extrapolated)) constitutes more than 20%, the AUC_{inf} was considered less reliable.

The half-life T₂ was calculated as In2/1_{z}, where 1_{z} was the elimination rate constant. The half-life was only calculated when at least 3 data points could be included. If the regression line resulted in an Rsq of less than 0.80, the results were not considered reliable.

After collection of the last blood sample/procedure, the animal was no longer part of the study and was terminated.

### Results

At Injection Site 1, after dosing with Formulation A (lactose) initially no reaction was observed. However, at Day 14, a soft swelling was observed (up to 10×15 mm, which was barely perceptible).

The day after dosing with Formulation B at Injection Site 2 (azacitidine, 50 mg), a hard swelling (up to 40×20 mm) was observed, which lasted for at least 28 days. Minimal to slight erythema was observed on Days 2 and 3 and again from Day 7. The day after dosing with Formulation B at Injection Site 4 (also azacitidine, 50 mg), a soft swelling (up to 10×10 mm, which was barely perceptible), which lasted until 11 clays after dosing (Days 23 to 33). Minimal erythema was observed from Days 23 to 28.

However, the day after dosing with Formulation B at injection Site 3 (azacitidine, 100 mg), a hard, well-defined swelling at the injection site (up to 55×30 mm, which lasted at least 28 days. Slight erythema was observed on Days 2 and 3 and again from Day 7.

Conversely, the day after dosing with Formulation D at Injection Site 5 (azacitidine 50 mg plus indomethacin 50 mg), no injection site reaction whatsoever was observed.

In terms of PK parameters, the single subcutaneous administration of 50 mg of coated azacitidine (Injection Site 4) showed a systemic exposure with prolonged release profile as shown in FIG. 5. The duration was 120 h and 47% of the exposure was observed within the first 12 h post administration.

The histopathology results demonstrated the following: Injection Site 2: moderate inflammation and moderate necrosis at Day 3 and mild inflammation and moderate necrosis at Day 7.

Injection Site 5 moderate inflammation and mild necrosis at Day 45 (3 days after injection) and minimal inflammation and minimal necrosis at Day 49 (7 days after injection).

It was concluded that subcutaneous administration of Formulation D (combination of mixed oxide coated azacitidine and mixed oxide coated indomethacin) caused less skin reaction than subcutaneous administration of Formulation B (mixed oxide coated azacitidine alone).

### Example 16

### Mini Pig Study II

A similar study to that described in Example 15 above was conducted on five minipigs. On the day of arrival, the animals were given a final number, using a randomisation scheme. The animals received a chip with a unique numerical code.

Approximately on week prior to star of treatment, two injection sites were marked on necks of minipigs in the same manner as described in Example 15 above

The animals' treatment schedule was as set out in Table 7 below. Vidaza® (Mylan), the commercial injectable formulation of azacitidine with a concentration of 25 mg/mL can be considered to provide an equivalent dose of ‘uncoated’ azacitidine,

TABLE 7

| | | | Dose | | |
|---|---|---|---|---|---|
| Animal/ | Injection | Formula- | Volume | Dose(s) (mg) | |
| Group | Site | tion | (mL) | Azacitidine | Indomethacin |
| 1 | 1 | B | 0.5 | 50 | 0 |
| | 2 | F | 0.5 | 50 | 12.5 |
| 2 | 1 | G | 0.5 | 50 | 2.5 |
| | 2 | D | 0.5 | 50 | 50 |
| 3 | 1 | E | 0.25 | 50 | 0 |
| | 2 | H | 0.25 | 50 | 6.25 |
| 4 | 1 | I | 0.25 | 50 | 1.25 |
| | 2 | Vidaza | 2 | 50 | 0 |
| 5 | 1 | Vidaza | 2 | 50 | 0 |
| | 2 | J | 0.25 | 50 | 25 |

Essentially the same treatment protocol as that described in Example 14 above was followed. For each animal subcutaneous injections of relevant formulations were administered at the above dose volumes on Day 1 (Injection Site 1 on each animal) and Day 8 (Injection Site 2 on each animal).

Observations were carried out in essentially the same manner as described in Example 14 above, with necropsy on Day 29.

The size of the inflammatory swelling in mm³ is presented in FIG. 6 for:
Animal 1/Injection Site 1 (50 mg azacitidine, lower concentration of particles; diamonds);
Animal 3/injection Site 1 (50 mg azacitidine, higher concentration of particles; triangles);
Animal 2/Injection Site 2 (50 mg azacitidine, lower concentration of particles+50 mg indomethacin; crosses); and
Animal 5/Injection Site 2 (50 mg azacitidine, higher concentration of particles+25 mg indomethacin; squares).

FIG. 6 clear shows the huge impact administration of azacitidine with indomethacin.

1. A pharmaceutical formulation that is useful in the treatment of myelodysplastic syndrome, comprising a plurality of particles suspended in an aqueous carrier system, which particles:
(a) have a weight-, number-, or volume-based mean diameter that is between amount 10 nm and about 700 μm; and
(b) comprise solid cores comprising azacitidine, or a pharmaceutically-acceptable salt thereof, coated, at least in part, by a coating of inorganic material comprising mixture of:
(i) zinc oxide; and
(ii) one or more other metal and/or metalloid oxides,
wherein the atomic ratio ((i):(ii)) is at least about 1:6 and up to and including about 6:1.

2. A formulation as claimed in claim 1, wherein the atomic ratio ((i):(ii)) is at least about 1:1 and up to and including about 6:1.

3. A formulation as claimed in claim 1, wherein the coated particles comprise:
(a) solid cores comprising azacitidine, or pharmaceutically-acceptable salt thereof; and
(b) one or more discrete layers surrounding said cores, said one or more discrete layers each comprising at least one separate mixture of zinc oxide and one or more other metal and/or metalloid oxides in a atomic ratio of between about 1:1 and about 6:1.

4. A formulation as claimed in claim 3, wherein the cores consist essentially of azacitidine, or a pharmaceutically-acceptable salt thereof.

5. A formulation as claimed in any one of the preceding claims, wherein the weight-, number-, or volume-based mean diameter of the particles is between amount 1 μm and about 50 μm.

6. A formulation as claimed in any one of the preceding claims, wherein more than one discrete layer of the mixture of oxides is applied to the core sequentially.

7. A formulation as claimed in claim 6, wherein between 3 and 10 discrete layers of the mixture of oxides are applied.

8. A formulation as claimed in any one of the preceding claims, wherein the total thickness of the mixed oxide coating is between about 0.5 nm and about 2 μm.

9. A formulation as claimed in any one of claims 6 to 8, wherein the maximum thickness of an individual discrete layer of mixed oxide coating is about 1 hundredth of the weight-, number-, or volume-based mean diameter of the core, including any other discrete layers that have previously been applied to the core.

10. A formulation as claimed in any one of the preceding claims, wherein the ratio of zinc oxide to other metal and/or metalloid oxides is between about 2:1 and about 5:1.

11. A formulation as claimed in any one of the preceding claims, wherein the one or more other metal and/or metalloid oxides are selected from aluminium oxide and/or silicon dioxide.

12. A formulation as claimed in any one of the preceding claims in the form of a sterile injectable and/or infusible dosage form.

13. A formulation as claimed in claim 12 in a form that is administrable via a surgical administration apparatus that forms a depot formulation.

14. A process for the preparation of a formulation as defined in any one of the preceding claims, wherein the coated particles are made by applying the layer(s) of mixed oxide coating material to the cores, and/or previously-coated cores, by atomic layer deposition.

15. A process as claimed in claim 14, wherein:
(i) solid cores are coated with a first discrete layer of mixed oxide coating material;
(ii) the coated cores from step (i) are then subjected to a deagglomeration process step;
(iii) the deagglomerated coated cores from step (ii) are then coated with a second discrete layer of mixed oxide coating material;
(iv) repeating steps (ii) and (iii) to obtain the required number of discrete layers.

16. A process as claimed in claim 15, wherein the deagglomeration step that takes place between applications of coatings comprises sieving.

17. A process as claimed in claim 16, wherein the sieving comprises vibrational sieving.

18. A process as claimed in claim 17, wherein the vibrational sieving comprises controlling a vibration probe coupled to the sieve.

19. A process as claimed in claim 16, wherein the sieving comprises sonic sifting.

20. A process for the preparation of a formulation as defined in any one of claims 1 to 13 wherein the coated particles are mixed with the carrier system after coating.

21. An injectable and/or infusible dosage form comprising a formulation as defined in any one of claims 1 to 13 contained within a reservoir that is connected to, and/or is in association with, an injection or infusion means.

22. A dosage form as claimed in claim 21, which is a surgical administration apparatus that forms a depot formulation.

23. A dosage form as claimed in claim 21 or claim 22, wherein coated particles as defined in any one of claims 1 to 12 and the carrier system are housed separately, and in which admixing occurs prior to and/or during injection or infusion.

24. A formulation as defined in any one of claims 1 to 13, or a dosage form as defined in any one of claims 21 to 23, for use in the treatment of myelodyspastic syndrome.

25. The use of a formulation as defined in any one of claims 1 to 13, or a dosage form as defined in any one of claims 20 to 22, for the manufacture of a medicament for the treatment of myelodysplastic syndrome.

26. A method of treatment of myelodyspastic syndrome, which method comprises administering a formulation as defined in any one of claims 1 to 13, or a dosage form as defined in any one of claims 21 to 23, to a patient in need of such treatment.

27. A formulation for use as claimed in claim 24, a use as claimed in claim 25 or a method as claimed in claim 26, wherein the myelodyspastic syndrome is selected from the group refractory anaemia, refractory anaemia with ringed sideroblasts, refractory cytopenia with multilineage dysplasia, refractory cytopenia with multilineage dysplasia and ringed sideroblasts, refractory anemia with excess blasts, refractory anaemia with excess blasts in transformation, unclassified myelodysplastic syndrome and myelodysplastic syndrome associated with isolated del (5q).

28. A formulation for use, a use or a method as claimed in any one of claims 24 to 27, wherein, following injection, the formulation provides a depot formulation from which azacitidine is released over a period of time that is between 3 days and about 3 weeks.

29. A formulation for use, a use or a method as claimed in claim 28, wherein the total exposure for azacitidine is at least about 50% of the total exposure obtained from a dosing regimen comprising administering 75 mg/m² of body surface area over seven consecutive days by injection or infusions of azacitidine.

30. A formulation for use, a use or a method as claimed in claim 29, wherein the exposure in terms of the average area under the concentration vs. time curve up to infinite time is 960±458 ng*h/mL.

31. A formulation for use, a use or a method as claimed in any one of claims 28 to 30, wherein the average maximum concentration observed in the plasma is less than that obtained from a dosing regimen comprising administering 75 mg/m² of body surface area over seven consecutive days by injection or infusions of azacitidine.

32. A formulation for use, a use or a method as claimed in claim 31, wherein the average maximum concentration observed in the plasma is between about 200 and about 700 ng/mL.

33. A formulation as claimed in any one of claims 1 to 13, a dosage form as claimed in any one of claims 21 to 23, or a formulation for use, a use or a method as claimed in any one of claims 24 to 32, wherein the close of azacitidine or pharmaceutically-acceptable salt thereof (calculated as the free compound) is in the range of about 200 mg to about 1000 mg per m² of body surface area.
